(19) United States
(12) Patent Application Publication
(10) Pub. No.: US 2021/0348783 A1
(43) Pub. Date: Nov. 11, 2021

(54) DEVICE FOR DEBILITATING AIRBORNE INFECTIOUS AGENTS AND ASSOCIATED SYSTEMS AND METHODS

(71) Applicant: UV Health Group, Portland, OR (US)

(72) Inventors: Neil Thomas Adkins, Portland, OR (US); Steven Arthur Daniel, Hillsboro, OR (US); Norman James Adkins, Portland, OR (US); Stephen Andrew Batti, Portland, OR (US)

(21) Appl. No.: 17/313,929

(22) Filed: May 6, 2021

Related U.S. Application Data
(60) Provisional application No. 63/021,293, filed on May 7, 2020.
(60) Provisional application No. 63/125,238, filed on Dec. 14, 2020.

(51) Int. Cl. F24F 8/22; A61L 2/10; F24F 3/167

(52) U.S. Cl. CPC F24F 8/22; A61L 2/10; A61L 2202/25; A61L 2202/11; F24F 3/167

(57) ABSTRACT

Devices for debilitating airborne infectious agents in an enclosed space, and associated systems and methods, are disclosed herein. A representative device includes a housing extending in an axial direction from a first to a second end. The housing includes an internal chamber defining an airflow pathway extending in the axial direction. The internal chamber includes a surface, at least a portion of which is reflective. The housing also includes a first opening toward the first end and positioned to allow air flow into the internal chamber, and a second opening toward the second end and positioned to allow air flow out of the internal chamber. The device can also include an air mover in fluid communication with the internal chamber and a light emitting diode (LED) component contained in the internal chamber. The LED component is positioned to emit ultraviolet (UV) radiation into the internal chamber toward the reflective surface.

### CROSS-REFERENCE TO RELATED APPLICATION(S)

The present application claims priority to the following U.S. Provisional Applications: U.S. Provisional Application No. 63/021,293 filed May 7, 2020 and U.S. Provisional Application No. 63/125,238 filed Dec. 14, 2020, the disclosures of which are incorporated herein by reference in their entireties.

### TECHNICAL FIELD

The present technology relates to debilitating airborne infectious agents. More specifically, embodiments of the present technology include systems and methods for using ultraviolet radiation to debilitate airborne infectious agents.

### BACKGROUND

Ultraviolet (UV) radiation has been shown to be an effective method for debilitating infectious agents (e.g., killing, disabling, inactivating (e.g., sterilizing), neutralizing, or otherwise preventing infectious agents from engaging in harmful activity). In particular, shortwave UV-C radiation (having a wavelength range from about 100 nanometers (nm) to about 280 nm) can kill and/or inactivate infectious agents by damaging their deoxyribonucleic acid (DNA) or their Ribonucleic Acid (RNA). Photons from the UV radiation impact the infectious agent and cause chemical reactions as energy is transferred to the infectious agent. In particular, photons impacting the DNA or RNA of the infectious agent cause reactions between two molecules of thymine, thereby causing thymine dimers to form. In turn, the thymine dimers can render the infectious agent incapable of replicating. The greater the exposure to UV radiation, the more thymine dimers are formed, and therefore the greater the chance the infectious agent will be unable to replicate.

Air disinfection can be accomplished through several methods. First, UV-C radiation can be targeted at the upper regions of an enclosed space. However, this method involves the risk of human exposure and may therefore be implausible to include in a standard-height room when occupied. Alternatively, a space can be directly and completely irradiated when the space is not occupied. These methods cannot reduce the spread of infectious diseases during a critical period of human interaction when the space is occupied. In a third method, air can be irradiated as it passes through enclosed air-circulation systems for heating and cooling the space, including while the space is occupied. However, this method is limited by the efficacy of the air-circulation systems as well as their ability to be integrated into the space. Further, this method can be limited to times when additional heating or cooling is required, often to the exclusion of times when the space is occupied. Accordingly, there remains a need for improved devices and techniques for disinfecting the air in enclosed, occupied spaces.

### BRIEF DESCRIPTION OF THE DRAWINGS

FIG. 1 is a partially schematic isometric view of a device for debilitating airborne infectious agents in accordance with some embodiments of the present technology.

FIG. 2 is a partially schematic cross-sectional view of a device for debilitating airborne infectious agents in accordance with some embodiments of the present technology.

FIGS. 3A and 3B are front and bottom views, respectively, of a device for debilitating airborne infectious agents in accordance with some embodiments of the present technology.

FIG. 3C is a partially schematic cross-sectional view of a device for debilitating airborne infectious agents in accordance with further embodiments of the present technology.

FIG. 4 is an isometric view of the device of FIG. 1 incorporated into an overhead fixture in accordance with some embodiments of the present technology.

FIG. 5 is an isometric view of the device of FIG. 1 incorporated into a fixture in fluid communication with a room in accordance with some embodiments of the present technology.

FIG. 6 is an isometric view of the device of FIG. 4 having fins positioned in an internal chamber in accordance with various embodiments of the present technology.

FIG. 7 is an isometric view of a device for debilitating airborne infectious agents incorporated into an overhead fixture in accordance with some embodiments of the present technology.

FIG. 8 is an isometric view of a device for debilitating airborne infectious agents incorporated into an overhead fixture in accordance with further embodiments of the present technology.

FIG. 9 is an isometric view of a device for debilitating airborne infectious agents incorporated into a wall-mounted fixture in accordance with some embodiments of the present technology.

FIG. 10 is an isometric view of a device for debilitating airborne infectious agents incorporated into another overhead fixture in accordance with further embodiments of the present technology.

FIG. 11 is an isometric view of a device for debilitating airborne infectious agents incorporated into a wall-mounted light fixture in accordance with some embodiments of the present technology.

FIG. 12A is an isometric view of a device for debilitating airborne infectious agents incorporated into a vertical light fixture in accordance with some embodiments of the present technology.

FIG. 12B is an isometric view of a device for debilitating airborne infectious agents incorporated into another vertical light fixture in accordance with some embodiments of the present technology.

FIG. 12C is a partially transparent view of a device generally similar to the vertical light fixture of FIG. 12B.

FIG. 13 is an illustration of a device for debilitating airborne infectious agents in a configuration that underwent testing in accordance with some embodiments of the present technology.

FIG. 14 is an isometric view of an elevator car having multiple devices for debilitating airborne infectious agents incorporated therein in accordance with some embodiments of the present technology.

FIG. 15A is an isometric view of a device for debilitating airborne infectious agents incorporated into a wall-mounted fixture in accordance with some embodiments of the present technology.

FIG. 15B is an isometric internal view of the device of FIG. 15A, configured in accordance with some embodiments of the present technology.

FIG. 16 is an isometric internal view of a device for debilitating airborne infectious agents incorporated into a modular fixture in accordance with some embodiments of the present technology.

FIG. 17A is an isometric view of a device for debilitating airborne infectious agents incorporated into a table fixture in accordance with some embodiments of the present technology.

FIG. 17B is a bottom view of the device of FIG. 17A, configured in accordance with some embodiments of the present technology.

FIG. 18 is a diagram of opposing UV light emitters for use within a device for debilitating airborne infectious agents in accordance with some embodiments of the present technology.

The drawings have not necessarily been drawn to scale. Similarly, some components and/or operations can be separated into different blocks or combined into a single block for the purpose of discussion of some of the implementations of the present technology. Moreover, while the technology is amenable to various modifications and alternative forms, specific implementations have been shown by way of example in the drawings and are described in detail below.

### DETAILED DESCRIPTION

### Overview

Devices for debilitating airborne infectious agents, and associated systems and methods, are disclosed herein. A representative device includes a housing extending in an axial direction from a first end to a second end. The housing includes an internal chamber defining an airflow pathway that extends in the axial direction. The internal chamber includes at least one surface, at least a portion of which is reflective and/or includes a reflective material disposed thereon. The housing also includes a first opening at or near the first end and a second opening at or near the second end. The first end is positioned to allow air to flow into the internal chamber and the second end is positioned to allow air to exit the internal chamber. In some embodiments, the device also includes an air mover in fluid communication with the internal chamber. The air mover is positioned to direct airflow through the internal chamber. The device also includes one or more light emitting diodes (LEDs) contained in the internal chamber. The one or more LEDs are positioned to emit ultraviolet (UV) radiation into the internal chamber toward the reflective surface.

During operation, the device can be positioned in fluid communication with the air in a predefined space, such as an occupied room. The air mover forces air from the predefined space into the airflow pathway and through the internal chamber. As air flows through the internal chamber, it carries infectious agents (e.g., harmful bacteria and/or viruses) through one or more paths of the UV radiation. The one or more LEDs emit UV radiation in a first travel path across the internal chamber towards the reflective surface. Some of the photons in the UV radiation thereby impact the infectious agents to debilitate the infectious agents (e.g., kill, disable, inactivate (e.g., sterilize), neutralize, or otherwise prevent the infectious agents from engaging in harmful activity). Some of the photons in the UV radiation that do not impact an infectious agent impinge on the reflective surface and are reflected back across the internal chamber in a second travel path. The reflected photons may impact an infectious agent to debilitate the infectious agents. As a result, some of the reflected UV radiation is effectively recycled to further disinfect the air flowing through the internal chamber. The disinfected air then flows out of the second opening and, for example, back into the predefined space.

In some embodiments, the device can include a controller that toggles the components of the device between multiple modes of operation. For example, the device can include a disinfecting mode, a power saving mode, and a chamber-cleaning mode. In the disinfecting mode, the device operates as described above to debilitate airborne infectious agents. The controller can toggle the device into the disinfecting mode when a room is occupied, or shortly before, to actively debilitate infectious agents in the room. In the power saving mode, one or more of the components can consume less power and/or be turned off completely. The controller can toggle the device into the power saving mode after a room is unoccupied for a period of time. In some embodiments, the period can be long enough to allow the device to sufficiently disinfect the air in the room after occupation. Once disinfection is complete, the device can shift to a lower power consumption rate while the room remains unoccupied. In the chamber-cleaning mode, the speed of the air mover can be increased to blow contaminants (e.g., dust, dirt, condensed moisture, other particulates, bugs, and/or other contaminants) out of the internal chamber. The controller can toggle the device into the chamber-cleaning mode on a set schedule to maintain clean surfaces on the LED component and/or the reflective surface.

For ease of reference, the device is sometimes described herein with reference to specific directions and/or orientations (e.g., top and bottom, upper and lower, and/or upwards and downwards relative to the spatial orientation of the embodiments shown in the figures. It is to be understood, however, that the device can be moved to, and used in, different spatial orientations without changing the structure and/or function of the disclosed embodiments of the present technology.

Many embodiments of the technology described below may take the form of computer- or machine- or controller-executable instructions, including routines executed by a programmable computer or controller. Those skilled in the relevant art will appreciate that the technology can be practiced on computer/controller systems other than those shown and described below. The technology can be embodied in a special-purpose computer, controller or data processor that is specifically programmed, configured or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described below may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

### Description of the Figures

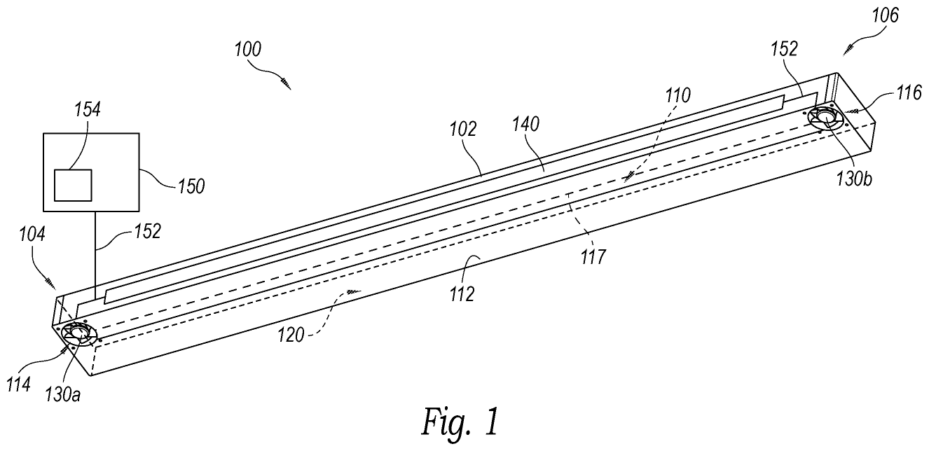

FIG. 1 is an isometric view of a representative device 100 for debilitating airborne infectious agents in accordance with some embodiments of the present technology. As illustrated, the device 100 has a housing 102 extending in an axial direction from a first end 104 to a second end 106. The housing 102 has an internal chamber 110 extending in the axial direction and defining an airflow pathway 117. The internal chamber 110 includes at least one surface 112 internal to the housing 102, at least a portion of which is reflective, as well as a first opening 114 toward the first end 104 and a second opening 116 toward the second end 106. Each of the first and second openings 114 and 116 is positioned to allow air to flow in and/or out of the internal chamber 110. In some embodiments, for example, air flows into the internal chamber 110 through the first opening 114, flows axially through the internal chamber 110 along the airflow pathway 117, and flows out of the internal chamber 110 through the second opening 116.

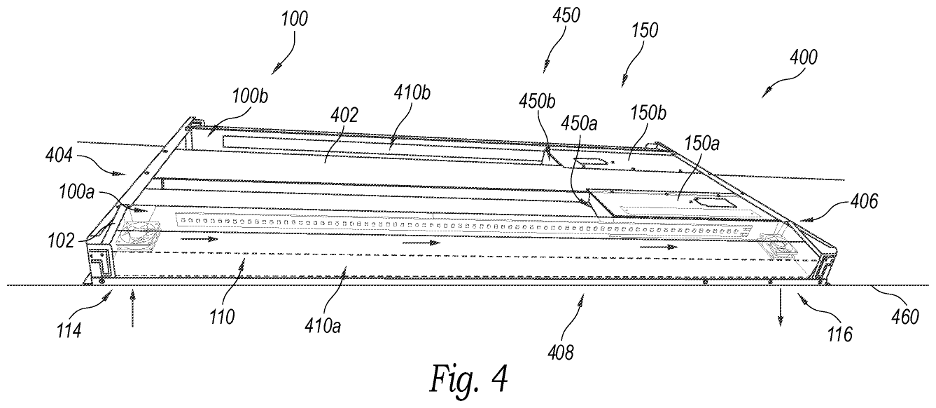

Although the device 100 is discussed herein primarily as sized to be incorporated into an overhead fixture (e.g., as shown in FIG. 4), one of skill in the art will understand that the scope of the present technology is not so limited. For example, the device 100 can also take the form of a stand-alone modular housing, incorporated into various other fixtures, incorporated into an existing HVAC or other airflow system, and/or incorporated into various other suitable systems. Further, although each of the components of the device 100 are illustrated as contained within a single housing (housing 102), the device 100 can have a further modularized construction as suitable to aid in incorporating the device 100 into various systems.

In some embodiments, the internal chamber 110 is bounded, at least in part, by a reflective material 120, such that the entire surface 112 of the internal chamber 110 is reflective. In some embodiments, a first portion of the internal chamber 110 is bounded by the reflective material 120 while a second portion is not (or is covered by a non-reflective material), such that only the first portion is reflective. In still other embodiments, the internal chamber 110 is partially or entirely bounded by a non-reflective material, but the reflective material 120 is nevertheless in optical communication with the internal chamber 110 (e.g., if the reflective material 120 is a backing layer on an interiorly-facing transparent layer of glass or another suitable material).

In the illustrated embodiment, the first opening 114 and the second opening 116 are each positioned on a bottom surface of the housing 102. In various other embodiments, the first and/or second openings 114, 116 can be positioned on the top surface, side surface, and/or the end surfaces. In addition, although depicted as positioned immediately adjacent the first and second ends 104, 106, it will be understood that the first and/or second openings 114, 116 or can be positioned further away from the first end 104 and the second end 106, respectively.

As further illustrated in FIG. 1, the device 100 also includes one or more air movers 130 in fluid communication with the airflow pathway 117. In the illustrated embodiment, for example, the device 100 includes a first air mover 130a positioned in the first opening 114 and a second air mover 130b positioned in the second opening 116, each in fluid communication with the airflow pathway 117 and/or the surrounding environment through the first and second openings 114, 116, respectively. In various other embodiments, the air movers 130 can be internal or external to the housing 102 and positioned a distance away from the first and second openings 114, 116 and/or the device 100 can include only a single air mover 130. For example, the device 100 can include only the first air mover 130a, which can push air into the airflow pathway 117 through a fluid communication channel (e.g., a vent channel) connected to the first opening 114. In some embodiments, the device 100 can be integrated into another system that causes airflow (e.g., an air-circulation system in a building, an air-circulation system in an airplane or bus, or any other suitable system), allowing the device 100 to omit one or both of the air movers 130.

The air movers can comprise one or more axial fans (e.g., a typical blade fan, a bladeless fan, or other suitable axial fan), centrifugal fans, tangential fans, pumps, jets, or other suitable devices. In some embodiments, as explained in further detail below, one (or both) of the air movers 130 can be configured to operate in multiple modes (e.g., at multiple speeds).

As further illustrated in FIG. 1, the device 100 also includes a light emitting diode component 140 (“LED component” 140) that includes one or more LEDs positioned to emit ultraviolet (UV) radiation into the internal chamber 110 towards the surface 112. Depending on the embodiment, the LED component 140 can be (or can include) a single LED, an array of LEDs, an LED panel, or various other suitable LED components. In the illustrated embodiment, the LED component 140 is an array of LEDs that extends in the axial direction from a position adjacent the first opening to a position adjacent the second opening. The LED component emits UV radiation into the internal chamber 110 to debilitate infectious agents in the air by killing, inactivating (e.g., sterilizing by causing thymine dimers to form in the DNA and/or RNA of the infectious agents), or otherwise neutralizing the infectious agents. The UV radiation can be UV-C light having a wavelength in a wavelength range from about 200 nanometers (nm) to about 300 nm, from about 240 nm to about 280 nm, from about 260 nm to about 280 nm, or from about 265 nm to about 275 nm.

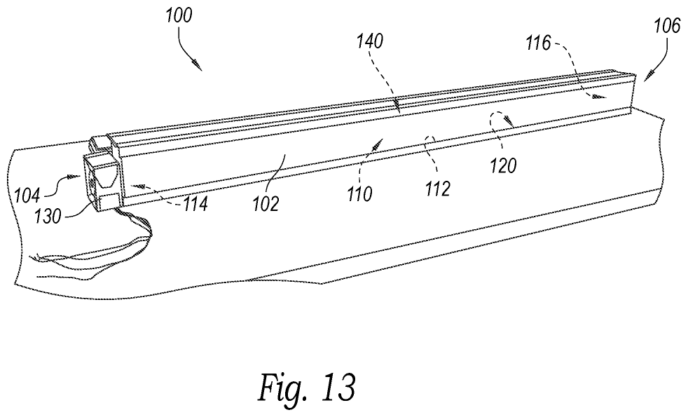

The UV radiation impacts particles and infectious agents in the air moving along the airflow pathway 117, and can thereby debilitate the impacted infectious agents. For example, as discussed below with reference to FIG. 13, test results have shown that UV-C radiation with a wavelength in a wavelength range between about 265 nm and about 275 nm can kill more than 99% of a SARS-CoV-2 virus (the virus that causes COVID-19) within the internal chamber 110. As a result, air is disinfected as it flows along the airflow pathway 117 in the internal chamber 110. Accordingly, the device 100 can be positioned or coupled in fluid communication with a space to draw air in from the space, disinfect the air, and expel disinfected air back into the space.

As further illustrated in FIG. 1, the device 100 also includes a controller 150 in communication with various components of the device 100 through one or more communication links 152. In the illustrated embodiment, the controller 150 is in communication with the LED component 140, the first air mover 130a, and the second air mover 130b through the communication links 152. The communication links 152 allow the controller 150 to control the power supply to and/or control the operation of each of the components. In some embodiments, the communication links 152 allow the controller 150 to toggle one or more of the components between multiple modes of operation. In some embodiments, for example, the device 100 can include a disinfecting mode, a power saving mode, and a chamber-cleaning mode.

The controller 150 can place the device 100 in the disinfecting mode, for example, to help reduce the infectious agents in an occupied room and/or help disinfect the air in a room shortly before and/or after it is occupied. In the disinfecting mode, the air movers 130 can operate at a speed configured to cycle the air in a space a preset number of times per hour while the LED component 140 emits UV radiation into the internal chamber 110. The operation speed of the air movers 130 can be selected to maximize the debilitation of the infectious agents in the space by striking a balance between a higher cycle rate and a higher UV exposure time for air within the chamber. While a higher operation speed can cycle the air in the room more times per hour, higher speeds can also result in some infectious agents surviving in the output air (e.g., due to the lower time of exposure to the UV radiation within the internal chamber 110). In contrast, a lower cycle rate can help ensure more infectious agents are debilitated as air flows through the internal chamber 110, but can lead to a buildup of infectious agents in the space because the air is cycled less frequently. Accordingly, the operation speed can be balanced based on the desired number of cycles, the size of the space, and/or various other relevant considerations. In a representative embodiment, for example, the air movers 130 can cause the air in an 8-foot by 8-foot by 10-foot space to cycle through the internal chamber 110 of the device 100 at least four times per hour.

The controller 150 can place the device 100 in the power saving mode when a room is unoccupied, to avoid redundantly circulating clean air. In the power saving mode, one or more of the components of the device can be adjusted to consume less power than in the disinfecting mode. For example, in the power saving mode, the operation speed of the air movers 130 can be reduced, or the air movers 130 can be turned off completely (e.g., on an intermittent or continuous basis). Similarly, the intensity of the LED component 140 can be reduced (e.g., resulting in less UV radiation being emitted into the internal chamber 110), or the LED component 140 can be turned off completely (e.g., on an intermittent or continuous basis). In some embodiments, in addition to reducing the power consumption of the device 100, the power saving mode can extend the life of the components of the device 100. For example, by turning the LED component 140 off, the power saving mode can extend the life of the LEDs in the LED component.

In many conventional devices using UV radiation to disinfect air, the device will include an advanced antimicrobial filter to aid in the disinfection and prevent most particles from flowing through the device (e.g., a HEPA filter, a filter made from a fibrous material, and/or a filter made from randomly oriented strands of fibers). However, these filters can be costly, often requiring maintenance multiple times per year. They are also often complicated to service, due to their inclusion in a closed, inconveniently located unit and/or the logistics of handling of potentially infectious, used filters. Further, they can impede the air flow significantly, requiring higher-powered air movers to move air through the device and to achieve the same cycle rate in a room. In part to address these shortcomings, some embodiments of the device 100 do not include a filter in the airflow pathway 117. As a result, the airflow pathway 117 is relatively unimpeded, requiring less power for the air movers to cycle air through the device 100; and the device 100 requires significantly less maintenance time and costs to change a filter and/or address filter-related issues. In some embodiments, as discussed in more detail below, the device 100 includes a screen that can prevent larger contaminants (e.g., insects or other large contaminants) from entering the internal chamber. In some such embodiments, the screen can be positioned in an easily accessed location (e.g., on the outside of the housing 102) for quick maintenance when necessary. Further, as discussed in more detail below, the screen can have a relatively large mesh rating that does not impede the airflow pathway 117 and requiring less maintenance throughout a year.

However, because in some embodiments the device 100 do not include an air filter, contaminants such as dust, dirt, condensed moisture, insects, and/or other particulates can accumulate on the reflective surface 112 of the internal chamber 110 and/or the active surface of the LED component 140. As the contaminants accumulate, they can block some of the UV radiation from being emitted and/or reflected, thereby lowering the efficacy of the device 100. To account for this accumulation, the controller 150 can place the device 100 in the chamber-cleaning mode to blow the contaminants out of the internal chamber 110. To do so, one or more of the air movers 130 can operate at a higher speed, thereby increasing the airflow velocities within the internal chamber 110 to blow the accumulated contaminants out of the internal chamber 110. The device 100 can be placed into the chamber-cleaning mode as often as necessary to maintain the efficacy of the device. In various embodiments, for example, the controller 150 can place the device 100 in the chamber-cleaning mode after each period in the disinfecting mode, once per hour, once per day, once per week, once per month, or after any other suitable period. In some embodiments, after the internal chamber 110 has been cleaned, the device 100 reenters the disinfecting mode. In some embodiments, the controller 150 can place the device 100 in the disinfecting mode automatically after the completion of the chamber cleaning mode without any input from a user. In these embodiments, the controller 150 can help minimize the down time for the device 100 and thereby increase the efficacy of the device 100.

In various embodiments, the device 100 can include any suitable number of operational modes corresponding to varied operation of one or more of the components. For example, in addition to (or in lieu of) the modes of operation described above, the device 100 can include multiple disinfecting modes based on various air mover operation speeds (and therefore varied air exchange cycle rates); multiple power saving modes based on various air mover operation speeds and/or varied LED component intensities; and/or multiple chamber-cleaning modes.

In some embodiments, the controller 150 can be configured to toggle the components of the device 100 between operational modes according to a predetermined schedule. For example, the controller 150 can be configured to toggle the components of the device 100 into the disinfecting mode during common work hours for an office (e.g., between 8:00 AM and 6:00 PM), the power saving mode during non-common work hours, and the chamber-cleaning mode once per week. In some embodiments, the controller 150 can include a communications component 154 allowing the controller 150 to communicate with one or more external devices (e.g., over a Network, through short range radio frequency, or any other suitable communications link). For example, the controller 150 can be connected to a network that is in turn connected to multiple computing devices (e.g., smartphones, desktop computers, laptop computers, tablets, and/or other suitable computing devices), which can schedule blocks of time during which a space will be occupied. During the occupied blocks, the controller 150 can toggle the components of the device 100 to the disinfecting mode. In some embodiments for which blocks of occupation time are scheduled, the controller 150 can be configured to toggle the components of the device 100 to the disinfecting mode for a preset length of time before occupation and/or to continue the disinfecting mode a preset length of time after occupation to clean the space before and after occupation. In some embodiments, the controller 150 can be connected to a control panel (e.g., a wall mounted panel, table mounted panel, or other suitable component) to receive inputs for toggling between operational modes. In some embodiments, the control panel can include one or more safety protections (e.g., password protection, key protection, user warning, or other suitable security measure) to prevent the device 100 from being turned off unintentionally. In some embodiments, the controller 150 can be connected to a switch (e.g., a wall mounted switch) to receive inputs for toggling between operational modes. In some embodiments, the controller 150 can be connected to one or more occupancy sensors (e.g., infrared sensors, ultrasonic sensors, microwave sensors, environmental sensors, and/or other suitable sensors). In these embodiments, the controller 150 can be configured to toggle the component(s) of the device 100 into the disinfecting mode when the sensors detect the space is occupied. Additionally, or alternatively, the controller 150 can be configured to toggle the component(s) to the power saving mode when the sensors do not detect an occupant within a set time period (e.g., for ten minutes, twenty minutes, thirty minutes, an hour, or any other suitable period).

In some embodiments, the device 100 can include additional sensors (not shown) that allow the controller 150 to account for various other environmental and/or operational factors. For example, the device can include an ambient light sensor, a humidity sensor, a temperature sensor, and/or various other suitable sensors. In embodiments that include an ambient light sensor, the controller 150 can control the system differently during the daytime than at night. For example, the controller 150 can adjust the brightness of a multi-color LED component 140 status-indicator light (not shown) and/or adjust a room-facing lighting component (see, e.g., FIG. 4, below). In embodiments that include a humidity sensor, the controller 150 can automatically adjust the power level of the LED component 140 to compensate for differing humidity levels to maintain a target (e.g., optimal) debilitation rate of the infectious agents. For example, the controller 150 can increase the power level of the LED component 140 in response to a high humidity level to compensate for UV radiation absorbed by the airborne water molecules. In embodiments that include a temperature sensor, the temperature sensor can measure the temperature at the surface of the LED component 140 and/or the ambient temperature in the air. In some embodiments, the controller 150 can then adjust the power levels of the components of the device 100 to maintain the LED component 140 at or around a predetermined temperature. The predetermined temperature can be selected to adjust (e.g., maximize) the radiation output and/or maximize the life span of the LED component 140.

In some embodiments, the device 100, e.g., the controller 150, can also include one or more non-volatile memory devices (not shown) to store usage metrics, maintenance metrics, environmental metrics, or various other suitable metrics. In some embodiments, individual components of the device 100 can include a memory device storing metrics specific to the component. Including an independent memory device for individual components allows the statistics for a component to be recorded on the component itself as components are switched and/or serviced (allowing, e.g., a replacement LED component 140 to record statistics specific to the replacement LED component 140 and separate from the statistics for the device 100). In some embodiments, for example, the LED component 140 includes a memory device that stores a serial number allowing the LED component 140 to be tracked back to a manufacturing source, and/or stores various relevant metrics for monitoring the health of the LED component (e.g., an original performance of the LED component 140, performance over time, the ambient air temperature during operation, the temperature of the surface of the LED component 140 during operation, the electrical current consumed by the LED component 140, the total usage (e.g., total time on), maintenance performed, and/or various other suitable metrics). In some embodiments, the controller 150 (or another suitable component in communication with the device) can use any of the stored metrics to report the current health of the device 100 and/or predict when the device 100 might fail or require service. In some embodiments, the controller 150 can also report the health status of the device 100 to an external network, allowing users to monitor the health of the device 100. In some embodiments, the controller 150 can send a maintenance report as soon as any of the components of the device 100 fail, thereby shortening the downtime of the device 100 and flagging the malfunction to users. In some embodiments, tracking statistics specific to components of the device 100 directly on or at the components can help ensure an accurate accounting of the lifespan and/or health of the components over time. For example, if an LED component 140 is recycled from a first device 100 to a second device 100, the second device 100 can access the memory on the LED component 140 to access statistics for the LED component in assessing and/or reporting the health of the device 100.

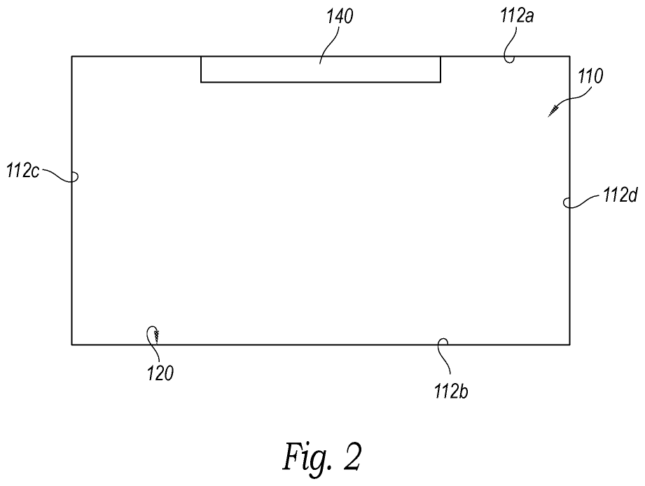

FIG. 2 is a partially schematic cross-sectional views of the internal chamber 110 of the device 100 of FIG. 1 configured in accordance with several embodiments of the present technology. The internal chamber 110 can include any suitable number of surfaces 112 (or a single continuous surface 112) and the components therein can be arranged in any of a variety of suitable configurations. In the embodiment illustrated in FIG. 2, for example, the internal chamber 110 includes four surfaces 112a-d, including a first (e.g., top) surface 112a, a second (e.g., bottom) surface 112b, a third (e.g., left side) surface 112c, and a fourth (e.g., right side) surface 112d. The LED component 140 is carried by the first surface 112a and positioned to emit UV radiation across the internal chamber 110 toward the second surface 112b. In addition, the second surface 112b includes a reflective material 120 (e.g., polished aluminum, Teflon, steel, stainless steel, or another suitable material) positioned to reflect the UV radiation incident on the second surface back across the internal chamber 110. UV radiation that impacts an infectious agent or particle may be absorbed and/or refracted to another wavelength of light, while UV radiation that does not impact an infectious agent or particle reaches the second surface 112b. UV radiation that reaches the second surface 112b is reflected, thereby “recycling” the “unused” UV radiation to impact infectious agents in the internal chamber. In some embodiments, the third and fourth surfaces 112c, 112d can also include a reflective material. Accordingly, a significant portion of the overall surface area of the internal chamber 110 is configured to reflect and recycle UV radiation that has not already impacted an infectious agent or other particle. As a result, a larger portion of the generated UV radiation debilitates infectious agents than in systems that do not contain reflective surfaces to recycle the UV radiation, thereby improving the energy efficiency and efficacy of the device 100.

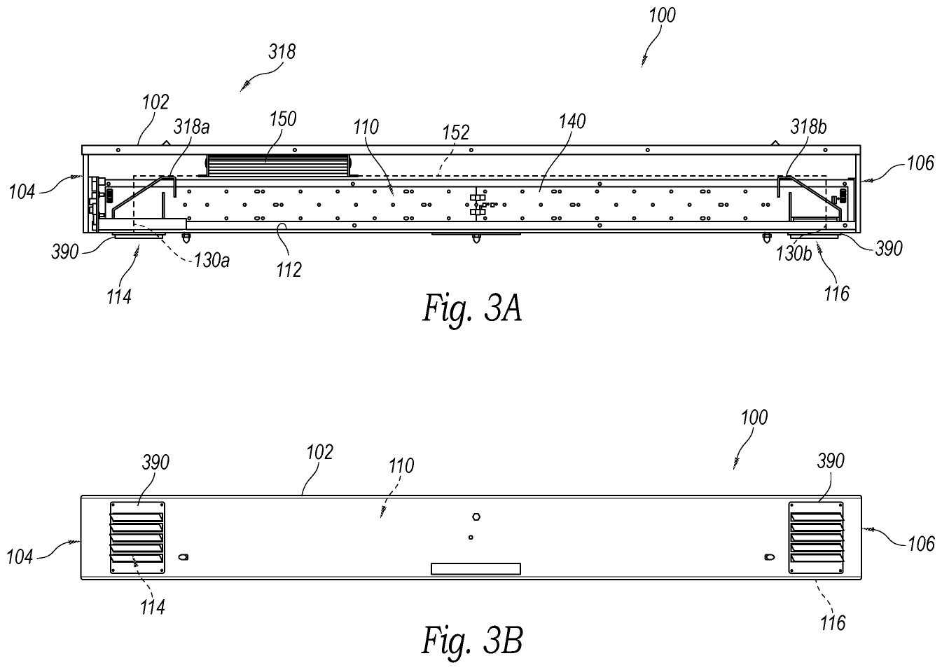

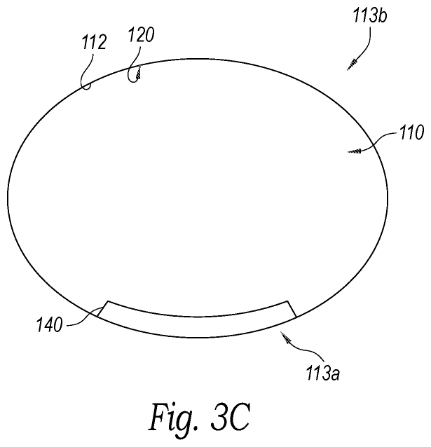

FIGS. 3A and 3B are partially schematic front and bottom views, respectively, of a device 100 of the type shown in FIG. 1, configured in accordance with some embodiments of the present technology. As illustrated in FIG. 3A, the device 100 includes generally similar features to those discussed above with reference to FIG. 1. For example, the device 100 includes the housing 102 extending in an axial direction from the first end 104 to the second end 106. The housing includes the internal chamber 110 with the at least one surface 112 internal to the housing 102, at least a portion of which is reflective, as well as the first opening 114 toward the first end 104 and the second opening 116 toward the second end 106. Each of the first and second openings 114, 116 is positioned to allow air to flow in and/or out of the internal chamber 110. The device 100 also includes the first air mover 130a positioned within the first opening 114, the second air mover 130b positioned within the second opening 116, and the LED component 140 positioned to emit ultraviolet (UV) radiation towards the at least one surface 112 in the internal chamber 110. The device 100 also includes the controller 150 in communication with the first air mover 130a, the second air mover 130b, and/or the LED component 140 through communication links 152 (shown schematically). For purposes of illustration, the controller 150 is shown at the same location as an associated power supply. One of skill in the art will understand that the controller 150 can be positioned in any other suitable location. For example, in some embodiments, the controller 150 is positioned within the housing 102 adjacent one of the first and second ends 104, 106. In another example, the controller 150 can be positioned external to the housing 102.

In the illustrated embodiment, the device 100 also includes baffles 318 (referred to individually as a first baffle 318a and a second baffle 318b) positioned to block UV radiation from exiting the internal chamber 110, thereby improving the safety of the device 100. In the illustrated embodiment, the baffles 318 are positioned partially between the LED component 140 and the first and second openings 114, 116 within the internal chamber 110. In various other embodiments, one or both of the first and second baffles 318a, 318b can be positioned fully between the LED component 140 and the first and second openings 114, 116 within the internal chamber 110, within the first and second openings 114, 116, and/or over an external surface of the first and second openings 114, 116.

In some embodiments, one or both of the first and second baffles 318a, 318b are operably connected to the controller 150 and/or another biasing device. In these embodiments, the controller 150 and/or other biasing device can deliver a biasing voltage to charge one or both of the first and second baffles 318a, 318b. As discussed above, the charge can draw the nucleus of an infectious agent towards the surface of the infectious agent. Once on the surface, the nucleus can be more easily destroyed by the UV radiation within the internal chamber 110, thereby aiding in debilitating the infectious agents.

As illustrated with reference to FIG. 3B, the device 100 can also include louvers 390 carried by the housing 102 over the first and second openings 114, 116. The louvers 390 can create a desired airflow current in a room in which the device 100 is deployed to improve circulation of disinfected air throughout the room. In some embodiments, the louvers 390 are adjustable, allowing a user to adapt the louvers 390 to the room in which the device 100 is deployed. In some embodiments, the one or both of the louvers 390 are operably connected to the controller 150 and/or another biasing device to charge the louvers 390. The charge can draw the nucleus of an infectious agent towards the surface of the infectious agent before the infectious agent enters the internal chamber 110. In some embodiments, the louvers 390 can be made from an antibacterial material. For example, in some embodiments, the louvers 390 can have a copper surface.

FIG. 3C is a partially schematic cross-sectional view of the internal chamber 110 of the device 100 of FIG. 3A configured in accordance with several embodiments of the present technology. As illustrated in FIG. 3C, the internal chamber 110 includes a generally smooth, continuous surface 112 (e.g., the internal surface of an ellipsoid) in cross-section. A first portion 113a of the surface 112 carries the LED component 140, while a second portion 113b of the surface 112 includes the reflective material 120. The LED component 140 emits UV radiation into and across the internal chamber 110 toward the second portion 113b, which then reflects (and thereby recycles) the unused UV radiation. In some embodiments, the first portion 113a and the second portion 113b comprise the entirety of the surface 112, allowing all the surface area that is not carrying the LED component 140 to be a reflective. In other embodiments, the first portion 113a and the second portion 113b comprise only a subset of the surface 112. For example, the first portion 113a can be a bottom portion of the surface while the second portion 113b can be a top portion opposite the bottom portion, leaving the sides of the surface remaining.

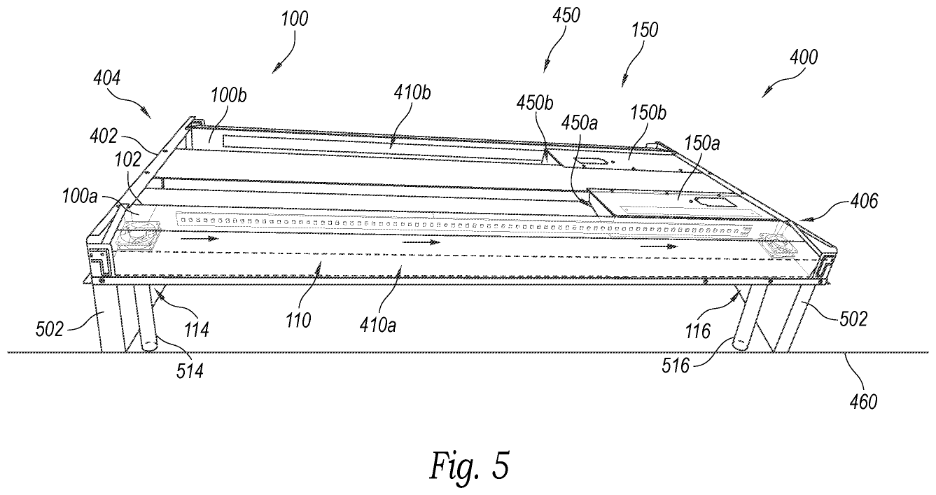

FIGS. 4 and 5 are isometric views of devices 100 (e.g., of the type shown in FIG. 1) incorporated into a fixture 400 in accordance with various embodiments of the present technology. As illustrated in FIG. 4, the fixture 400 can include a fixture housing 402 extending axially from a first end 404 to a second end 406 opposite the first end 404. The fixture housing 402 also includes one or more mounting portions 410 configured to receive one or more devices 100. In the illustrated embodiment, the fixture 400 includes two mounting portions 410a, 410b positioned on opposite transverse sides of the fixture housing 402, each having a device 100 mounted thereon (referred to individually as a first device 100a and a second device 100b). In other embodiments, the one or more mounting portions 410 can be positioned in the center of the fixture housing 402 and/or can extend in varying directions.

As further illustrated in FIG. 4, the fixture 400 can also include controller mounting locations 450 positioned to carry one or more controllers 150. In the illustrated embodiments, the fixture 400 includes a first controller 150a carried by a first controller mounting location 450a and a second controller 150b carried by a second controller mounting location 450b. The first controller 150a is in communication with the components of the first device 100a, while the second controller 150b is in communication with the components of the second device 100b. In some embodiments, the fixture 400 can include a single controller mounting location 450 carrying a single controller 150 in communication with the components of any number of devices 100. That is, in some embodiments, a single controller can be in communication with two or more devices to control and/or power the device 100 components.

In the embodiment illustrated in FIG. 4, the fixture 400 is an overhead fixture positioned with the bottom surface 408 generally flush with a ceiling 460 of a room. In this embodiment, the first and second openings 114, 116 of the device housing 102 of each device 100 can be oriented towards the room, thereby establishing a direct fluid communication with the room. Accordingly, when each device 100 is operated to disinfect the air in the room, air can be pulled into the internal chamber 110 directly from the room, disinfected, and expelled directly back into the room.

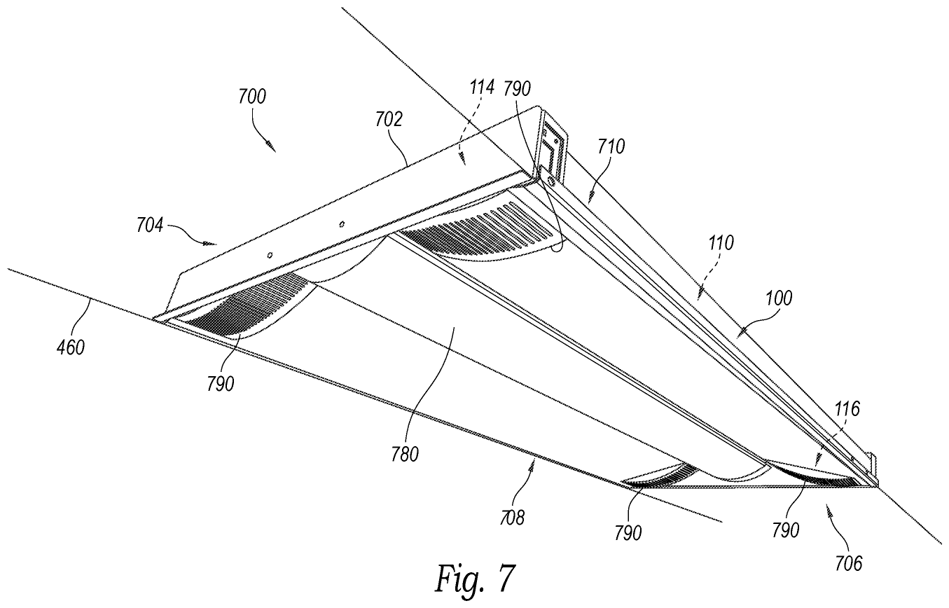

In various embodiments, the fixture 400 can be sized to replace an overhead ceiling tile, an overhead light, an overhead vent, and/or other overhead fixtures. In some embodiments, the fixture 400 can be integrated with various other devices having other functionalities. For example, as discussed in more detail below with reference to FIG. 7, the fixture 400 can integrated with an appropriate lighting source such that the fixture 400 can completely replace an overhead light without removing a light source from the room.

In still other embodiments, the fixture 400 can be spaced apart from the room altogether, for example as illustrated in FIG. 5. In the embodiment illustrated in FIG. 5, the fixture 400 is carried by risers 502, thereby positioning the fixture 400 a distance D above the ceiling 460. The first opening 114 is in fluid communication with the room through a first air channel 514 (e.g. an air duct, shaft, pipe, tubing, open air channel, or other suitable air channel). Similarly, the second opening 116 is in fluid communication with the room through a second air channel 516. Spacing the fixture 400 apart from the room allows the fixture 400 to be easily integrated into any space, since the fixture 400 need only be in fluid communication with the space and does not need to be sized and/or configured to replace existing components of the space.

Although illustrated as extending to/from the ceiling 460 in FIG. 5, the first and second air channels 514, 516 can have other suitable positions and/or orientations in other embodiments. For example, the first air channel 514 can be positioned to maximize the intake of infectious agents below the ceiling (e.g., on a wall or post to draw air in from a position below head-level in the room), while the second air channel 516 can be positioned to maximize the circulation of disinfected air (e.g., directed into the room at the ceiling level). Further, although illustrated as constrained tubular components in FIG. 5, in some embodiments, one (or both) of the first and second air channels 514, 516 can comprise an unobstructed air flow pathway between the room and the first and second openings 114, 116. In these embodiments, the device relies on circulation between the space above the room and the room to circulate disinfected air.

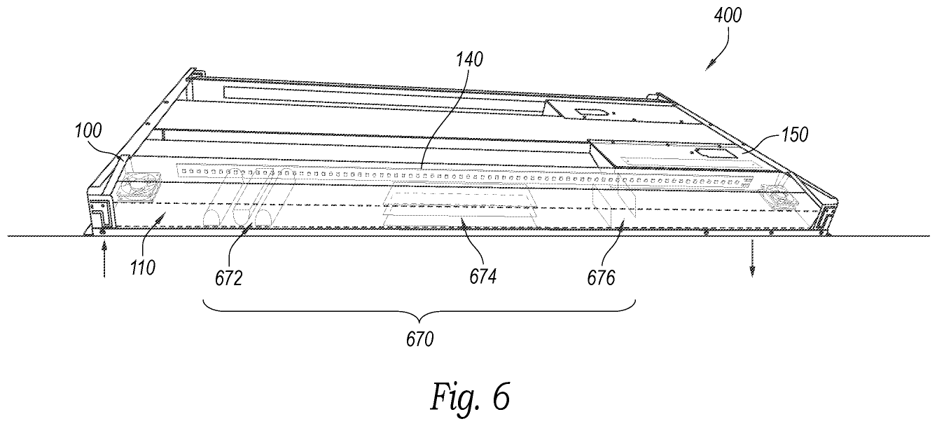

FIG. 6 is an isometric view of a representative device 100 having three examples of fins 670 positioned in the internal chamber 110 of the device 100 in accordance with various embodiments of the present technology. It will be understood that the device 100 can include one or more of the example fins 670, including one or more of the fins 670 illustrated in FIG. 6, as well as various other fin configurations designed to improve the functionality of the device.

For example, in some embodiments, the device 100 can include fins that are positioned to draw heat away from an active surface of the array of LED component 140. In these embodiments, the fins can contact the active surface and act as heat sinks for the LED component 140. As a result, the efficiency and/or output performance of the LED component 140 can be improved. In some embodiments, the fins are positioned to direct airflow within the internal chamber along one or more travel paths. The travel paths, e.g., serpentine paths, can be positioned directly in a UV radiation pathway, thereby increasing the chance infectious agents will be impacted by the UV radiation, and increasing the usage of UV radiation to disinfect the air. In some embodiments, the fins are positioned to create one or more axial streams within the internal chamber that travel directly through one or more UV radiation pathways. As a result, the axial streams increase the chances for infectious agents to be impacted by the UV radiation and increase the usage of UV radiation to disinfect the air. In some embodiments, the fins are positioned to create one or more areas of high pressure within in the internal chamber. The areas of high pressure have higher concentrations of infectious agents, and can accordingly be located in areas of higher intensity of UV radiation, thereby increasing the chance that infectious agents will be impacted by the UV radiation. In some embodiments, one or more of the fins can be electrically coupled to the controller 150. In these embodiments, the controller 150 can be configured to deliver a biasing voltage to charge one or more the fins. The charge can draw the nucleus of an infectious agent towards the surface of the infectious agent. Once on the surface, the nucleus can be more easily destroyed by the UV radiation, thereby aiding in debilitating the infectious agents. In some embodiments, the fins 670 are an extruded aluminum, Teflon, UV resistant plastic, or other material having suitable reflective, heat conductive, antimicrobial, UV resistance, and/or electrically conductive qualities.

In the illustrated embodiment, the internal chamber 110 includes alternating vertical fins 672, some carried by the bottom surface and some by the top surface of the internal chamber 110. The vertical fins 672 impede the airflow, causing the air to flow in alternating vertical directions as it moves generally in the axial direction through the internal chamber 110. In the illustrated embodiment, the internal chamber 110 also includes axial fins 674 that are carried by a surface 112 of the internal chamber 110 opposite the LED component 140. The axial fins 674 are generally flat plates extending in the axial direction generally parallel with the airflow pathway 117 (see FIG. 1). The axial fins 674 create one or more axial streams that are open to the LED component 140 and disposed in a travel path of the UV radiation. In the illustrated embodiment, the axial fins 674 create two axial streams (e.g., a first between the uppermost fin and the middle fin and a second between the middle fin and the lowermost fin), each axial stream within one or more pathways of the UV radiation emitted by the LED component 140. In some embodiments, the number of axial streams created by the axial fins 674 generally corresponds to the number of rows of LEDs in the LED component 140 to further direct airflow directly through UV radiation pathways. For example, an LED component 140 with three rows of LEDs can be matched by axial fins 674 that create three air streams, each air stream aligned with a row of LEDs. In the illustrated embodiment, the internal chamber 110 also includes transverse fins 676 alternately carried by a side surface opposite the LED component 140 and adjacent the LED component 140. The transverse fins 676 direct the airflow in a serpentine path, causing the air to flow at least partially in alternating transverse directions as it moves generally in the axial direction through the internal chamber 110.

FIG. 7 is an isometric view of a representative device 100 of the type shown in FIG. 1 incorporated into an overhead fixture 700 in accordance with some embodiments of the present technology. In the illustrated embodiment, the overhead fixture 700 includes a fixture housing 702 extending in an axial direction from a first end 704 to a second end 706, with a lower surface 708 positioned generally in the same plane as the ceiling 460. A visible light source 780 (e.g., LED panel, fluorescent lights, or any other suitable light source) extends in the axial direction in a central portion of the fixture housing 702.

As further illustrated in FIG. 7, the fixture housing 702 also includes a mounting portion 710 extending in an axial direction on a transverse side of the fixture housing 702. The device 100 carried by the mounting portion 710 and positioned with the first and second openings 114, 116 oriented towards the lower surface 708 to establish fluid communication with the room. Louvers 790 are carried by the fixture housing 702 and positioned to at least partially cover each of the first and second openings 114, 116. The louvers 790 can create a desired airflow current in the room to improve circulation of disinfected air throughout the room. In some embodiments, the louvers 790 are adjustable, allowing the louvers 790 to adapt to the room in which the overhead fixture 700 is deployed. In some embodiments, the fixture housing 702 can also include screens (not shown) positioned to at least partially cover the first openings. In some embodiments, for example, the screens can be carried by the backside of the louvers 790 between the louvers 790 and the first openings 114. The screens can at least partially prevent dust and other large contaminants from entering the internal chamber 110. In some embodiments, the screens can have a mesh rating (e.g., openings per square inch) of between about 5×5 and about 30×30, between about 12×12 and about 20 by 20, or of about 15×15. As a result, the device 100 can require fewer cycles of the chamber-cleaning mode discussed above to maintain efficacy. However, because the screens are external and do not include a fine mesh, fibrous materials, randomly oriented fibrous materials, and/or interior filters, they require minimal cleaning and effort to maintain. That is, unlike a filter, the screens do not require costly and complicated maintenance to maintain and/or replace. In some embodiments, the screens can include a copper surface. Copper has been shown to possess antimicrobial properties, allowing the screens to effectively prevent dust and other large contaminants from entering the internal chamber 110 without introducing a surface for infectious agents to collect on. In some embodiments, the screens (whether formed from copper or another conductive material) can be coupled to a charge delivery device (e.g., the controller 150 (FIG. 1) or another suitable charge delivery device). The charge applied to the screens can help attract dust and other large contaminants, further improving the efficacy of the screens.

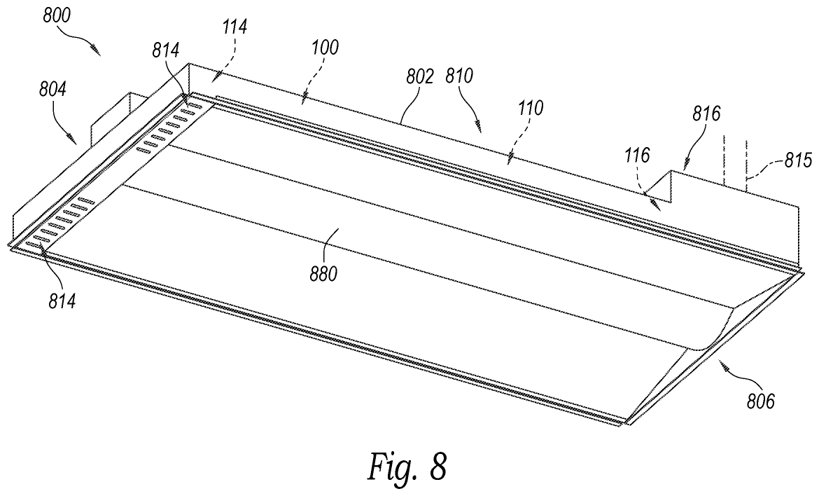

FIG. 8 is an isometric view of a representative device of the type shown in FIG. 1 incorporated into an overhead fixture 800 in accordance with further embodiments of the present technology. Elements of the overhead fixture 800 are generally similar to the overhead fixture 700 discussed above with reference to FIG. 7. For example, the overhead fixture 800 includes a fixture housing 802 extending in an axial direction, a light source 880 extending in the axial direction in a central portion of the fixture housing 802, and a mounting portion 810 positioned on a transverse side of the fixture housing 802.

In the illustrated embodiment, however, the mounting portion 810 includes first openings 814 at a bottom surface of the fixture housing 802 on a first transverse side of the fixture housing 802, and second openings 816 at the bottom surface of the fixture housing 802 on a second transverse side of the fixture housing 802. Further, in the illustrated embodiment, the fixture housing 802 carries two devices 100 (referred to individually as first device 100a and second device 100b) connected in series by one or more connecting elements 815 (e.g., internal air ducts). The first openings 814 of the mounting portion 810 are in fluid communication with the first opening 114 of the first device 100a while the second openings 816 are in fluid communication with the second opening 116 of the second device 100b. Air flows into the first device 100a through the first openings, travels axially through the first device 100a, transversely through the connecting elements 815, axially through the second device 100b, and back into the room. In some embodiments, the fixture housing 802 carries a single device 100 with the first openings 814 in fluid communication with the first opening 114 of the device 100 and the second openings 816 are in fluid communication with the second opening 116 of the device 100. In some embodiments, the device has a serpentine shape, extending the entire axial length of the housing 802 twice. In other embodiments, the housing 802 can include one or more connecting elements (e.g., internal air ducts) connecting the device 100 to the first and second openings 814, 816.

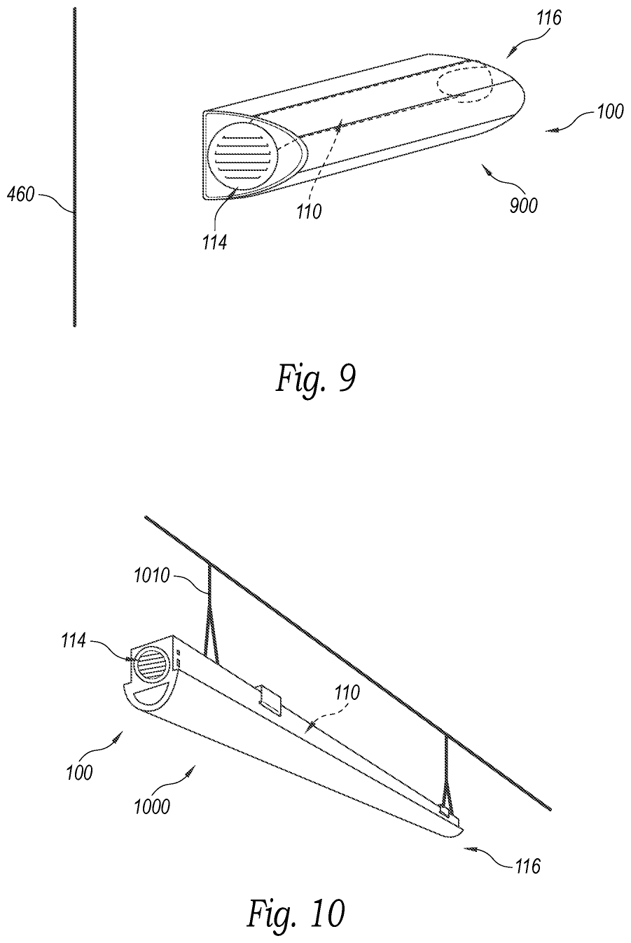

FIG. 9 is an isometric view of a device 100 of the type shown in FIG. 1 incorporated into a wall-mounted fixture 900 in accordance with some embodiments of the present technology. In the illustrated embodiment, the wall-mounted fixture 900 carries the device 100, and the internal chamber 110 of the device 100 extends in the axial direction from the first opening 114 to the second opening 116. The wall-mounted fixture 900 can be mounted to a wall 960 in a space to disinfect the air in the space bounded by the wall 960. In some embodiments, the wall-mounted fixture 900 can be mounted near an external power source (e.g., a wall outlet). In some embodiments, the wall-mounted fixture 900 can include one or more batteries (not shown), allowing the wall-mounted fixture 900 to be portable between spaces and/or used in spaces without access to an external power source.

FIG. 10 is an isometric view of a device 100 of the type shown in FIG. 1 incorporated into another overhead fixture 1000 in accordance with further embodiments of the present technology. In the illustrated embodiment, the overhead fixture 1000 carries the device 100, and the internal chamber 110 of the device 100 extends in the axial direction from the first opening 114 to the second opening 116. In turn, the overhead fixture 1000 is carried by one or more hanging elements 1010, allowing the overhead fixture 1000 to be easily deployed in many spaces. In some embodiments, the overhead fixture 1000 can positioned near an external power source (e.g., a ceiling outlet or electrical connection). In some embodiments, the overhead fixture 1000 can include one or more batteries (not shown), allowing the overhead fixture 1000 to be portable between spaces and/or used in spaces without access to an external power source.

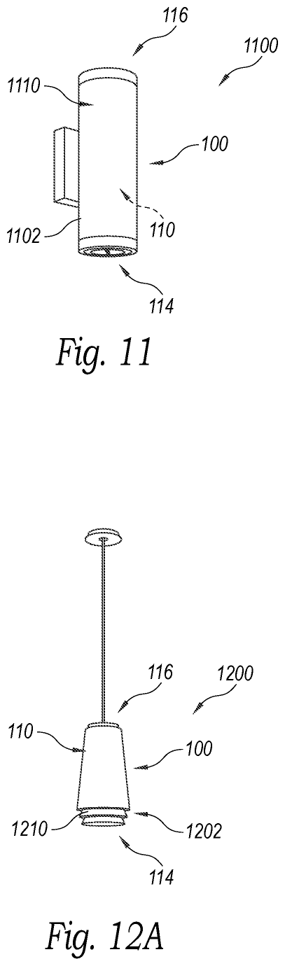

FIG. 11 is an isometric view of a device 100 of the type shown in FIG. 1 incorporated into a wall-mounted light fixture 1100 in accordance with some embodiments of the present technology. In the illustrated embodiment, the light fixture 1100 carries the device 100, and the internal chamber 110 of the device 100 extends in a vertical axial direction from the first opening 114 to the second opening 116. In this embodiment, the device 100 can cause a toroidal airflow pathway in a room, increasing the overall circulation of disinfected air throughout the room and increasing the circulation of infectious agents through the device 100. As further illustrated in FIG. 11, an exterior surface 1102 of the light fixture 1100 can include a visible light source 1110, allowing the light fixture 1100 to replace and/or supplement other light sources in a space.

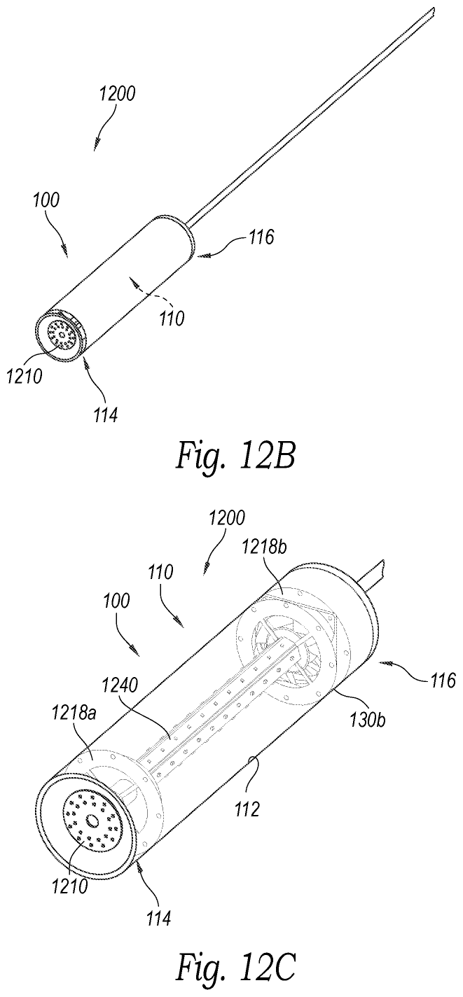

FIGS. 12A and 12B are isometric views of a device 100 of FIG. 1 incorporated into differently-shaped vertical light fixtures 1200 in accordance with some embodiments of the present technology. In the illustrated embodiments, the light fixture 1200 is generally similar to the light fixture 1100 FIG. 11. For example, the light fixture 1200 carries the device 100 with the internal chamber 110 extending in a vertical axial direction from the first opening 114 to the second opening 116. Accordingly, the device 100 can cause a toroidal airflow pathway in a room, increasing the overall circulation of disinfected air throughout the room and increasing the circulation of infectious agents through the device 100. However, in the illustrated embodiment, a bottom surface 1202 of the light fixture 1200 (e.g., as opposed to the side exterior surface 1102 of FIG. 11) can include a visible light source 1210, allowing the light fixture 1200 to replace and/or supplement other light sources in a space.

FIG. 12C is a partially transparent isometric view of the vertical light fixture 1200 of FIG. 12B, illustrating additional details in accordance with some embodiments of the present technology. In the illustrated embodiment, the internal chamber 110 of the device 100 includes an air mover 130 adjacent the second opening 116 and in fluid communication with the internal chamber 110. The device 100 also includes an LED component 1240 extending in the vertical axial direction from a first position adjacent the first opening 114 to a second position adjacent the air mover 130. As illustrated in FIG. 12C, the LED component 140 can be a central column with LED strips positioned to emit UV radiation radially outwardly towards a surface 112 lining the internal chamber 110. As described in detail above, at least a portion of the surface 112 can include a reflective material (e.g., polished aluminum, Teflon, steel, stainless steel, or another suitable material). As discussed in more detail above, the reflective material can reflect photons that do not impact an infectious agent back across the internal chamber 110. In some embodiments, the surface 112 can be a cylindrical surface with a substantially continuous curve. In some embodiments, the surface 112 can include an internal geometry with two or more sub surfaces. For example, the surface 112 can have a hexagonal tube shape, with six sub-surfaces (not shown) in the internal chamber 110.

As further illustrated in FIG. 12C, the device 100 can include baffles 1218 (referred to individually as a first baffle 1218a and a second baffle 1218b) positioned to prevent UV radiation from exiting the internal chamber 110. In the illustrated embodiment, the first baffle 1218a is positioned adjacent the first opening 114 while the second baffle 1218b is positioned between the air mover 130 and the second opening 116 within the internal chamber 110. Further, in the illustrated embodiment, the LED component 140 extends from the first baffle 1218a to the air mover 130. In various other embodiments, the baffles 1218 can be positioned in various other suitable locations to prevent UV radiation from leaving the internal chamber 110. For example, one or both of the first and second baffles 1218a, 1218b can be positioned over the first and second openings 114, 116, respectively, external to the internal chamber 110. In another example, the second baffle 1218b can be positioned between the LED component 140 and the air mover 130.

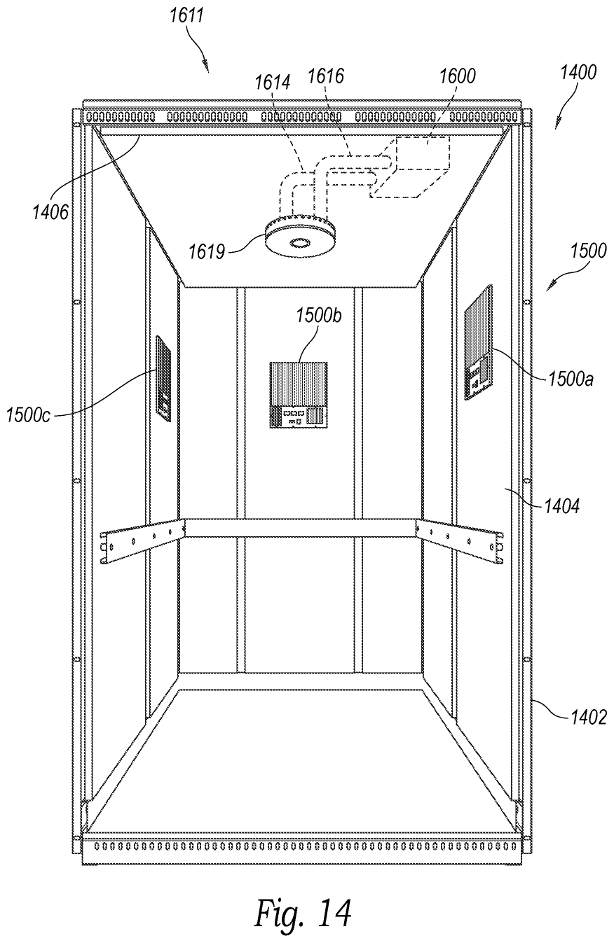

FIG. 13, described in further detail later, illustrates a test arrangement used to demonstrate the efficacy of devices used in both the arrangements described above, and the arrangements described below with reference to FIGS. 14-18.

FIG. 14 is an isometric view of an elevator car 1400 having multiple devices for debilitating airborne infectious agents incorporated therein in accordance with some embodiments of the present technology. As illustrated, the elevator car 1400 has a housing 1402 that includes three walls 1404, a door (shown cutaway), and a ceiling 1406. In the illustrated embodiment, the elevator car 1400 includes three wall-mounted fixtures 1500a-c each mounted to the walls 1404, and a modular fixture 1600 installed above the ceiling 1406.

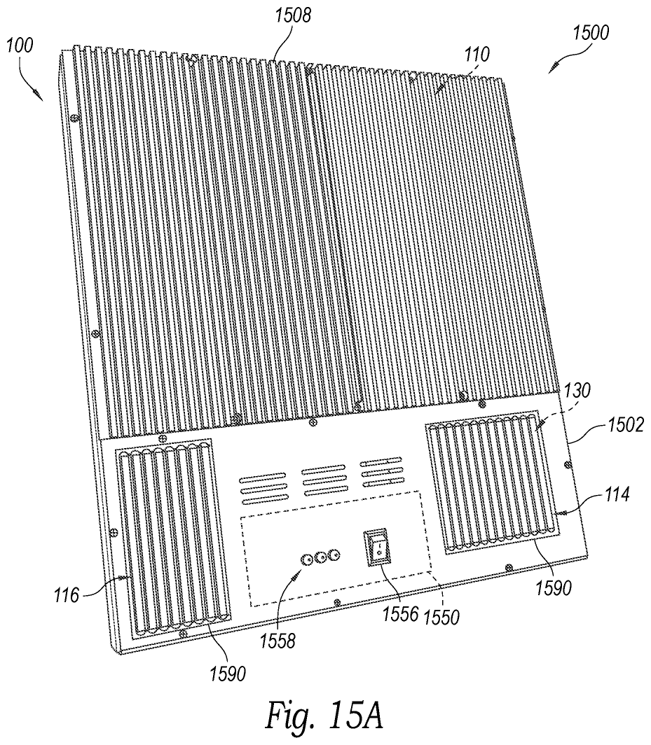

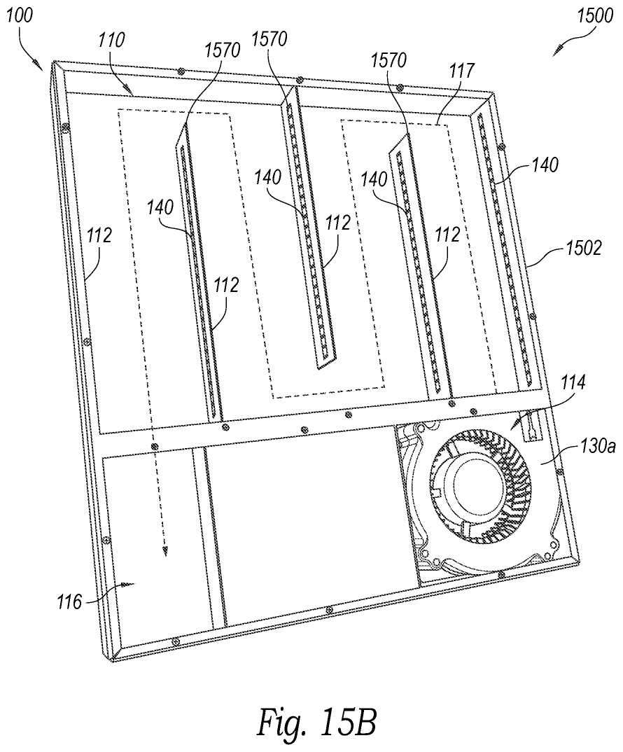

Each of the wall-mounted fixtures 1500a-c can be installed directly on or into a wall 1404. Accordingly, each of the wall-mounted fixtures 1500a-c can be in direct fluid communication with the space in the elevator car 1400. In the illustrated embodiment, each of the wall-mounted fixtures 1500a-c is installed at head-level in the elevator car 1400. In some embodiments, one or more wall-mounted fixtures 1500 can additionally, or alternatively, be installed at varying heights in the elevator car 1400. For example, the wall-mounted fixture 1500a can be at head level, the wall-mounted fixture 1500b can be above head level, and the wall-mounted fixture 1500c can be below head level. Additional details on examples of the wall-mounted fixtures 1500 are discussed below with reference to FIGS. 15A and 15B.

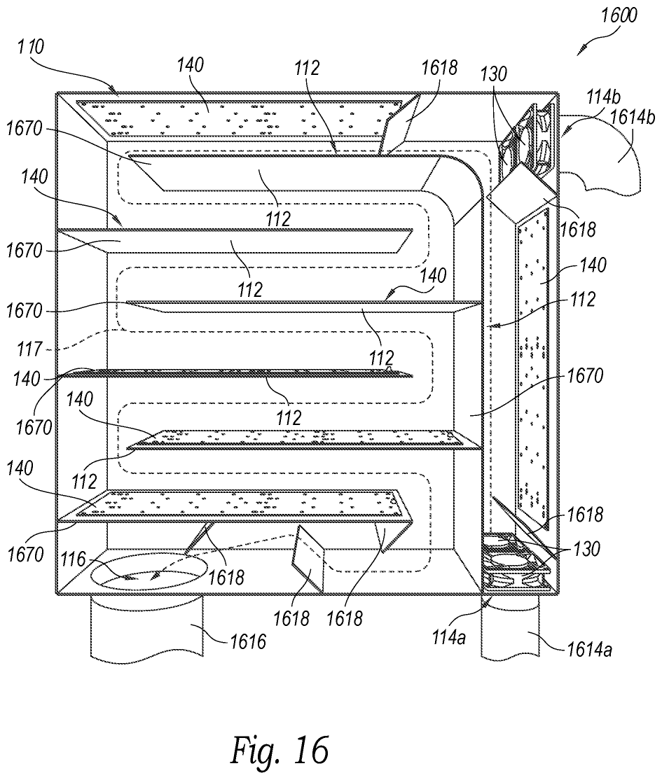

As further illustrated in FIG. 14, the modular fixture 1600 can be in fluid communication with the space in the elevator car 1400 through a duct system 1611. In the illustrated embodiment, the duct system 1611 includes a first air channel 1614 (e.g. an air duct, shaft, pipe, tubing, open air channel, or other suitable air channel), a second air channel 1616, and at least one connecting vent 1619. The first air channel 1614 is in fluid communication with an inlet of the modular fixture 1600, while the second air channel 1616 is in fluid communication with an outlet of the modular fixture 1600. The connecting vent 1619 establishes fluid communication between the space in the elevator car 1400 and the first and second air channels 1614, 1616, thereby establishing fluid communication with the inlet and outlet of the modular fixture 1600. Additional details of a representative modular fixture 1600 are discussed below with reference to FIG. 16.

Although FIG. 14 illustrates the disinfected space as being inside an elevator car 1400, one of skill in the art will understand that the fixtures 1500, 1600 discussed above can be deployed in various other spaces. For example, each of the fixtures 1500, 1600 can be deployed in one or more spaces in a doctor's office, emergency care building, hospital, an office building, a commercial building, a residential space, a public transportation space (e.g., within a train, subway car, bus, ferry, or other suitable mode of transportation), and/or in various other suitable spaces. Furthermore, additional or alternative fixtures can be employed in any given space. For example, the light fixture 1100 discussed above with reference to FIG. 11 can additionally, or alternatively, be deployed in the elevator car 1400 and/or any other suitable space. Further, although the elevator car 1400 is illustrated with four fixtures 1500, 1600, one of skill in the art will understand that fewer, or more, fixtures may be appropriate to disinfect the air in the space depending on the size of the space, the fixture chosen, and/or the desired cycle rate for the space.

FIGS. 15A is an isometric view of a device 100 of the type shown in FIG. 1 incorporated into the wall-mounted fixture 1500 in accordance with some embodiments of the present technology. As illustrated with reference to FIG. 15A, the wall-mounted fixture 1500 carries the device 100 within a housing 1502. The internal chamber 110 of the device 100 extends from the first opening 114 to the second opening 116, discussed in more detail below with reference to FIG. 15B, and an air mover 130 is positioned adjacent the first opening 114 to push air through the internal chamber 110.

As further illustrated in FIG. 15A, the housing 1502 of the wall-mounted fixture 1500 can include a grooved structure 1508 that acts as a heat sink for various components of the device 100. For example, the LED component 140 of the device can be thermally coupled to the grooved structure 1508 to dissipate heat throughout the grooved structure 1508. The large surface area of each of the grooves allows additional airflow of over the grooved structure 1508, thereby quickly dissipating heat into the ambient air.

As further illustrated in FIG. 15A, the housing 1502 of the wall-mounted fixture 1500 can include louvers 1590 positioned over the first and second openings 114, 116. The louvers 1590 can create a desired airflow current in a space in which the wall-mounted fixture 1500 is deployed to improve circulation of disinfected air throughout the space. In some embodiments, the louvers 1590 are adjustable, allowing a user to adapt the louvers 1590 to the space in which the wall-mounted fixture 1500 is deployed.

In the embodiment illustrated in FIG. 15A, the wall-mounted fixture 1500 also includes a power switch 1556 operably coupled to the controller 150 for the device 100. In the illustrated embodiment, the power switch 1556 allows a user to command the controller 150 to toggle the device 100 between an operational mode and a power saving mode (e.g., turned off). In some embodiments, the power switch 1556 allows a user to command the controller 150 to toggle the device 100 between additional operational modes. For example, the power switch 1556 can allow a user to command the controller 150 to toggle the device 100 into the chamber-cleaning mode. In the illustrated embodiment, the wall-mounted fixture 1500 also includes status indicators 1558. The status indicators 1558 can indicate the operational mode of the device 100, a health of various components of the device 100, a condition of the air exiting the device 100 (e.g., level of sanitization), and/or various other suitable indications. In some embodiments, the wall-mounted fixture 1500 can include a control panel in addition to, or instead of, the power switch 1556 and status indicators 1558. The control panel can allow a user to program an operation schedule for the device 100, command the controller 150 to toggle the device 100 between operational modes, view statistics for the device 100 and/or various components of the device 100, and/or perform various other suitable functions.

FIG. 15B is an isometric cutaway view of the wall-mounted fixture 1500 of FIG. 15A in accordance with some embodiments of the present technology. As illustrated in FIG. 15B, the internal chamber 110 of the device 100 can include fins 1570 that are similar to the vertical fins 672 and/or transverse fins 676 discussed above with reference to FIG. 6. The fins 1570 define a serpentine airflow pathway 117 through the internal chamber 110, thereby increasing the travel time within the internal chamber 110. Further, as illustrated in FIG. 15B, the device can include multiple LED components 140. Each LED component 140 can be carried by the housing 1502 of the wall mounted fixture 1500 and/or one of the fins 1570. In the illustrated embodiment, the device 100 also includes a surface 112 opposite each LED component 140 that can include a reflective material (e.g., polished aluminum, Teflon, steel, stainless steel, or another suitable material). As discussed in more detail above, the reflective material can reflect photons that do not impact an infectious agent back across the airflow pathway 117, thereby increasing the efficacy of the device 100.

In the illustrated embodiment, the device 100 includes an LED component 140 on a sidewall of the housing 1502 and each of the fins 1570. In some embodiments, the device 100 includes LED component(s) in various other positions. For example, the device 100 can include an LED component 140 carried by the backwall of the housing 1502, by the front wall of the housing 1502 (cut away in FIG. 15B), and/or on any other sidewall of the housing 1502. In another example, the device 100 can include LED components 140 positioned in an inverse configuration to the illustrated embodiment.

In some embodiments, the fins 1570 can act as heat sinks for the LED components 140 mounted thereon. In some embodiments, the fins 1570 can include a thermally conductive material in thermal communication with the grooved structure 1508 (FIG. 15A) of the wall-mounted fixture 1500. In some embodiments, one or more of the fins 1570 can be connected to a biasing voltage to charge the fins 1570. In some embodiments, the device 100 includes additional fins defining the serpentine airflow pathway 117. In some embodiments, the device 100 includes fewer fins defining the serpentine airflow pathway 117.

FIG. 16 is an isometric cutaway view of a device 100 of the type shown in FIG. 1 incorporated into a modular fixture 1600 in accordance with some embodiments of the present technology. Similar to the embodiments discussed above with reference to FIG. 15B, the illustrated device includes fins 1670 that define a serpentine airflow pathway 117 through the internal chamber 110, thereby increasing the travel time within the internal chamber 110. Individual fins 1670 (e.g., each fin 1670) carries an LED component 140 and the device includes a surface 112 opposite each LED component 140. Individual surfaces 112 (e.g., each surface 112) includes a reflective material (e.g., polished aluminum, Teflon, steel, stainless steel, or another suitable material) to reflect photons back across the airflow pathway 117.

As further illustrated in FIG. 16, the device 100 can include two first openings 114 (referred to individually as first opening 114a and first opening 114b), each having one or more air movers 130 positioned to pull air through the first openings 114 and push air through the internal chamber towards the second opening 116. The first opening 114a is connected to a first air channel 1614a, while the first opening 114b is connected to a first air channel 1614b. Each of the first air channels 1614a, 1614b can be a part of a duct system (e.g., the duct system 1611 discussed above with reference to FIG. 14) to place the first openings 114 in fluid communication with a space. Similarly, the second opening 116 is connected to a second air channel 1616, which can be connected to the duct system to place the second opening 116 in fluid communication with the space. In some embodiments, the device 100 can include additional, or fewer, first openings 114. In some embodiments, each of the first openings 114a, 114b is in fluid communication with a different space. For example, in some embodiments, the first air channel 1614a can connect to a first room of a building while the first air channel 1614b can connect to a second room of the building. In some such embodiments, the second air channel 1616 can connect to a duct system for the building, thereby pushing disinfected air into an established air distribution system.

In the illustrated embodiment, the device 100 also includes baffles 1618 positioned to block UV radiation from exiting the internal chamber 110. Accordingly, the baffles 1618 can help improve the safety of the device 100 by further ensuring that no harmful UV radiation exits the internal chamber 110. In the illustrated embodiment, the baffles 1618 are positioned within the internal chamber 110 and fully between all LED components 140 and the first and second openings 114, 116. In various other embodiments, the baffles 1618 can be located in different positions to block UV radiation from exiting the internal chamber.

In some embodiments one or more of the fins 1670 and/or one or more of the baffles 1618 can help draw heat away from the LED components 140. For example, the fins 1670 can thermally connect the LED components 140 to an external heat sink. In another example, the baffles 1618 can act as heat sinks and increase the surface area on which the LED components 140 can disperse heat. In some embodiments, one or more of the fins 1670 and/or one or more of the baffles 1618 can be connected to a biasing voltage to charge the fins 1670 and/or the baffles 1618.

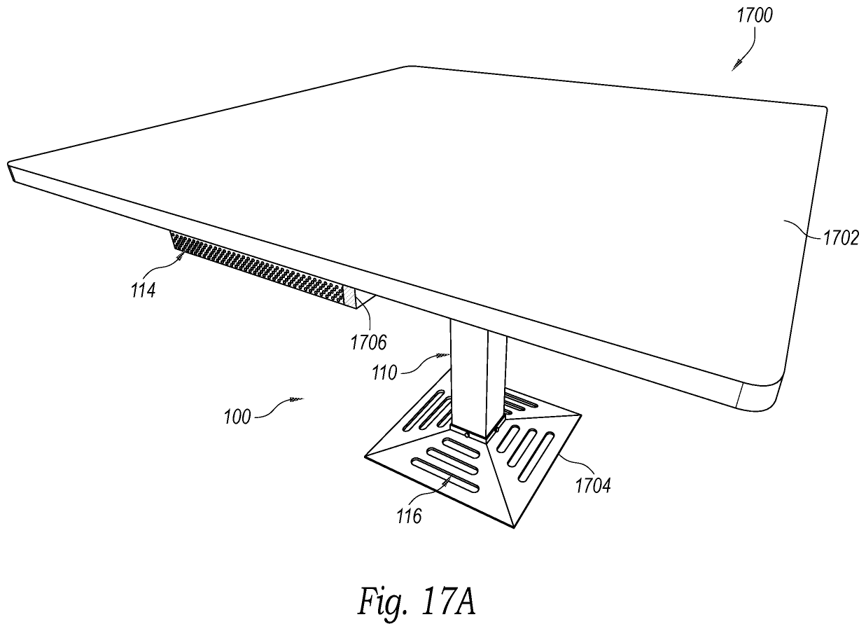

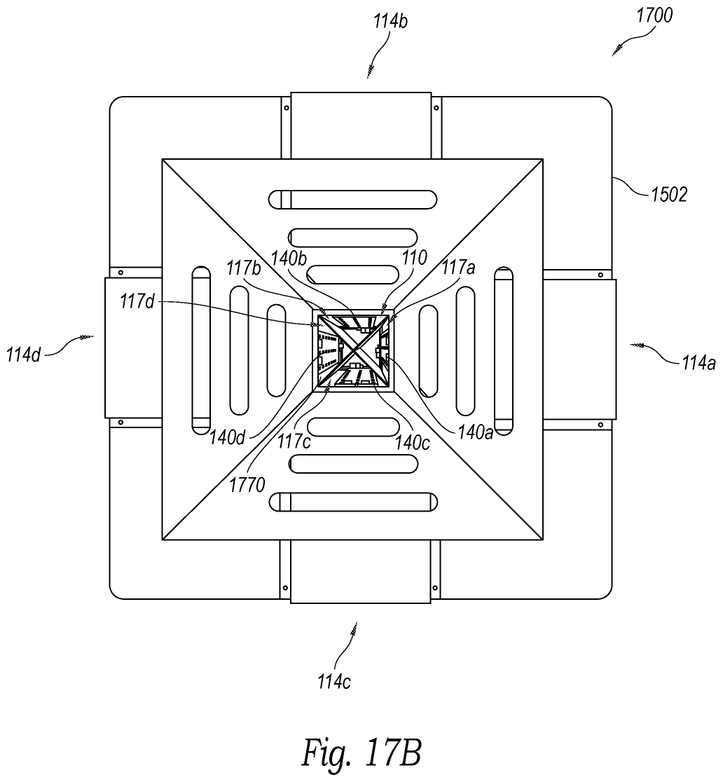

FIG. 17A is an isometric view of a device 100 of the type shown in FIG. 1 incorporated into a table fixture 1700 in accordance with some embodiments of the present technology. In the illustrated embodiment, the device 100 is incorporated into the upright central leg of the table fixture 1700. The internal chamber 110 of the device 100 extends in a vertical axial direction from one or more first openings 114 adjacent a table surface 1702 to one or more second openings 116 adjacent a foot 1704 of the table fixture 1700. In the illustrated embodiment, the device 100 includes a first opening 114 at each side of the table surface 1702 (FIG. 17B). In various embodiments, the device 100 can include multiple first openings 114 at each side of the table surface 1702, one first opening 114 for multiple sides of the table surface 1702 (e.g., at a ratio of 1:2 or any other suitable ratio including a single first opening), and/or a continuous first opening 114 along the entire perimeter of the table surface 1702. As illustrated, the one or more first openings 114 are positioned at table level, which is expected to contain a relatively high percentage of the contaminants from the air exhaled from people in the adjacent space. In the illustrated embodiment, the second opening 116 of the device 100 expels disinfected air down and circumferentially outward. Accordingly, the device 100 can draw air in from a region with a relatively high percentage of contaminants and push disinfected air outwards to diffuse into the room. In various other embodiments, the device 100 can include one or more air channels (not shown) in fluid communication with the second opening 116 to direct disinfected air in a desired direction and/or to a desired location.

As further illustrated in FIG. 17A, the table fixture 1700 can include one or more presence sensors 1706 (one shown) positioned to detect when a person is seated at or adjacent the table fixture 1700. The presence sensors 1706 can be operably connected to the device 100 to allow the device 100 to switch between operational modes based on whether an occupant is detected. For example, the device 100 can switch between the disinfecting mode and power saving mode discussed above with reference to FIG. 1 based on whether an occupant is detected.

FIG. 17B is a bottom view of the device 100 incorporated into the table fixture 1700 of FIG. 17A in accordance with some embodiments of the present technology. In the illustrated embodiment, the device 100 includes four first openings 114a-d and an internal fin 1770 that divides the internal chamber 110 into four flow paths 117a-d extending from the four first openings 114a-d to the second opening 116. The device 100 also includes four LED components 140a-d corresponding to the four flow paths 117a-d through the internal chamber 110. In some embodiments, the internal fin 1770 can be made from a reflective material (e.g., polished aluminum, Teflon, steel, stainless steel, or another suitable material). In some embodiments, the internal fin 1770 can be coated by the reflective material. As discussed in more detail above, the reflective material can reflect photons that do not impact an infectious agent back across the flow paths 117a-d, thereby increasing the efficacy of the device 100.

In some embodiments, the device 100 does not include the internal fin 1770. In these embodiments, the device includes a single flow path between the first openings 114a-d and the second opening 116. In some such embodiments, the internal chamber of the device 100 can include certain features generally similar to those discussed above with reference to FIGS. 1-3C. In some embodiments, the device 100 can include one or more additional, or alternative, internal fins 1770. For example, the internal fins 1770 can be configured as any of the fins 670 discussed above with reference to FIG. 6. In some embodiments, the internal fins 1770 can divide the internal chamber 110 into any other suitable number of flow paths. For example, the internal fins can divide the internal chamber 110 into two flow paths, three flow paths, five flow paths, or ten flow paths. In some embodiments, the internal fin 1770 can be connected to a biasing voltage to charge the internal fin.

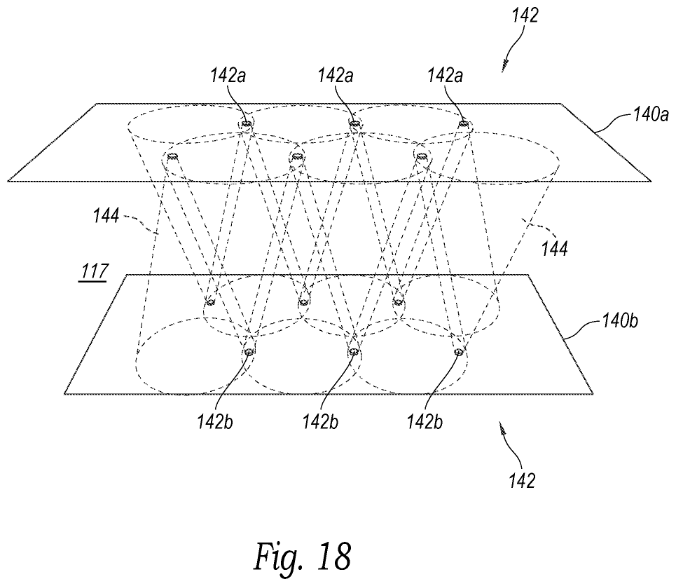

FIG. 18 is a partially schematic diagram of opposing LED components 140 for use with any of the devices described herein, in accordance with some embodiments of the present technology. As illustrated in FIG. 18, each of the LED components 140 can include a plurality of LEDs 142 that emit UV radiation along a radiation path 144. Each of the radiation paths 144 is generally conical in shape. That is, the area impacted by the UV radiation expands in a horizontal plane as the UV radiation travels farther in a vertical direction away from each of the LEDs 142. In some embodiments, LED components 140 can be positioned on opposing faces with their LEDs 142 staggered. The staggered position results in the radiation paths 144 more completely impacting the space between the LED components 140. For example, while first LEDs 142a along the upper LED component 140a do not emit UV radiation into the space immediately adjacent the first LEDs 142a, second LEDs 142b on the lower LED component 140b emit radiation into that space. Similarly, the first LEDs 142a along the upper LED component 140a emit UV radiation into the space immediately adjacent the second LEDs 142b along the lower LED component 140b. As a result, the opposing LED components 140 can help ensure the airflow pathway 117 has no dark sub-paths where a contaminant could travel through the internal chamber without being impacted by UV radiation.

### Experimental Results

Returning now to FIG. 13, FIG. 13 illustrates a device 100 according to still another representative embodiment of the present technology, and used to demonstrate the efficacy of representative devices disclosed herein. The illustrated device 100 includes the housing 102 extending from the first end 104 to the second end 106. In the illustrated embodiment, the first opening is positioned at the first end 104 of the housing 102 and an air mover 130 (e.g., a fan) is attached to the first end 104. Further, the second opening 116 is positioned at the second end 106 of the housing 102, with no air mover connected to the second opening. The internal chamber 110 extends from the first opening 114 to the second opening 116 and includes an LED component 140 in the form of an array of LEDs extending in the axial direction. The LEDs are configured to emit UV-C radiation having a wavelength in a wavelength range between about 265 nm and about 275 nm. A portion of the surface 112 of the internal chamber 110 is covered by a reflective material in the form of Teflon to reflect UV radiation within the internal chamber 110.

The device 100 depicted in FIG. 13 was subject to testing using live aerosolized SARS-CoV-2 virus (the virus causing COVID-19) in a highly controlled environment at an American university's Level 3 infectious disease laboratory. The conclusive results showed that the device 100 inactivated 99.6% of the infectious doses of the virus causing COVID-19 in the air in a single pass through the internal chamber 110. The results further showed that the device 100 could be operated continuously to pull room air into the internal chamber 110, proving that the device 100 can inactivate 99.999% of all the COVID-19 causing virus through multiple (e.g., two, three, four, five, ten, or any suitable number) passes through the internal chamber 110. The tested device 100 was sized and operated at a speed sufficient to disinfect the air of a 10-foot by 10-foot by 8-foot room of infectious agents, (e.g., those leading to COVID-19, influenza, the common cold, and/or other infectious diseases) within minutes.

### CONCLUSION

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. To the extent any materials incorporated herein by reference conflict with the present disclosure, the present disclosure controls.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In particular embodiments, devices of the type described herein can be incorporated into other systems and/or in other environments. For example, such devices can be positioned in an HVAC duct to debilitate infectious agents from air as the air enters and/or exits a room. In addition, certain aspects of the technology described in the context of particular embodiments may also be combined or eliminated in other embodiments. Furthermore, although advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

### EXAMPLES

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. A device for debilitating airborne infectious agents, the device comprising:
a housing extending in an axial direction from a first end to a second end, wherein the housing includes:
   an internal chamber defining an airflow pathway extending in the axial direction, the internal chamber having a surface, at least a portion of which is reflective;
   a first opening toward the first end, positioned to allow air flow into the internal chamber; and
   a second opening toward the second end, positioned to allow air flow out of the internal chamber; and
a light emitting diode (LED) contained in the internal chamber and positioned to emit ultraviolet (UV) radiation into the internal chamber toward the reflective surface.

2. The device of example 1, further comprising an air mover in fluid communication with the internal chamber of the housing and positioned to direct airflow.

3. The device of example 2 wherein:
the air mover is operable in multiple modes including an energy saving mode, a disinfecting mode, and a chamber-cleaning mode; and
the device further comprises a controller in communication with the air mover to toggle the air mover between modes.

4. The device of example 3 wherein the controller has instructions that, when executed:
receive a signal to toggle the air mover to the disinfecting mode; and
in response to receiving the signal, send a command to the air mover, the command causing the air mover to enter the disinfecting mode, wherein the disinfecting mode includes cycling air in a predefined space at least four times in an hour.

5. The device of any of examples 3 and 4 wherein the controller has instructions that, when executed, send a command to the air mover, the command causing the air mover to enter the chamber-cleaning mode, wherein the chamber-cleaning mode includes removing dust from the interior chamber.

6. The device of any of examples 3-5 further comprising a presence detection system in communication with the controller, wherein the presence detection system is positioned to detect occupation of a predefined space in fluid communication with the device.

7. The device of example 6 wherein the controller has instructions that, when executed:
receive a signal from the presence detection system, the signal indicating the occupation of the predefined space; and
in response to receiving the signal, send a command to the air mover, the command causing the air mover to enter the disinfecting mode, wherein the disinfecting mode is configured to cycle air in a predefined space at least four times in an hour.

8. The device of any of examples 1-7 wherein the surface of the internal chamber has a first portion and a second portion, and wherein a reflective material is disposed on the first portion of the surface and the LED is disposed on the second portion of the surface.

9. The device of any of examples 1-8 wherein the airflow pathway does not include a filter.

10. The device of any of examples 1-9 wherein:
the surface is a first surface; and
the internal chamber further comprises a second surface opposite the first surface; and
the LED is positioned to emit UV radiation in a direction from the second surface toward the first surface.

11. The device of any of examples 1-10 wherein the LED is configured to emit UV radiation having a wavelength in a wavelength range from 265 nanometers to 275 nanometers.

12. The device of any of examples 1-11, further comprising a copper screen positioned along the airflow pathway.

13. The device of example 12, further comprising charge delivery device coupled to the copper screen to provide a biasing voltage to the copper screen.

14. The device of any of examples 1-13, further comprising one or more fins positioned within the internal chamber.

15. The device of example 14, further comprising a charge delivery device coupled to the fins to provide a biasing voltage to the fins.

16. The device of any of examples 14 and 15 wherein the fins are positioned to draw heat away from an active surface of the array of LEDs.

17. The device of any of examples 14-16 wherein the LED emits UV radiation along one or more radiation travel paths, and wherein the fins are positioned to direct airflow within the airflow pathway in the internal chamber to intersect the one or more radiation travel paths.

18. The device of any of examples 14-17 wherein the fins are positioned to create an area of high pressure within the airflow pathway in the internal chamber, and wherein one or more LEDs are positioned to emit UV radiation directly into the area of high pressure.

19. A device for debilitating airborne infectious agents, the device comprising:
a housing having a first opening, a second opening spaced apart from the first opening, and an internal chamber defining an airflow pathway extending from the first opening to the second opening, wherein the internal chamber includes at least one internal surface having a reflective material;
a light emitting diode component (LED component) positioned to emit ultraviolet (UV) radiation into the internal chamber toward the reflective material on the internal surface.

20. The device of example 19, further comprising an air mover positioned in fluid communication with the internal chamber to force air through the internal chamber along the airflow pathway.

21. The device of any of examples 19 and 20, further comprising a controller in communication with at least one of the air mover or the LED component, the controller including a memory storing instructions that when executed cause the controller to toggle at least one of the air mover and the LED component between operational modes.

22. A method for operating a device for debilitating airborne infectious agents, the device including a housing with an internal chamber defining an airflow pathway, at least one air mover, and a light emitting diode (LED) component, the method comprising:
operating the device in a disinfecting mode, wherein, in the disinfecting mode:
   the LED component emits ultraviolet (UV) radiation into the airflow pathway at a first rate to debilitate infectious agents in the airflow pathway; and
   the air mover operates at a first speed to cycle air in fluid communication with the device at a predetermined flow rate;
toggling the device into a chamber-cleaning mode, wherein, in the chamber-cleaning mode the air mover operates at a second speed higher than the first speed to blow particulates out of the internal chamber.

23. The method of example 22, further comprising toggling the device into a power-saving mode.

24. The method of claim 23 wherein, in the power-saving mode:
the LED component emits the UV radiation into the airflow pathway at a second rate lower than the first rate; and
the air mover operates at a third speed lower than the first speed.

25. The method of claim 23 wherein, in the power-saving mode, the LED component and the air mover are not operated.

26. The method of any of examples 22-25, further comprising determining that a space in fluid communication with the device is unoccupied, and wherein the toggling the device into the power-saving mode is performed in response to determining that the space in fluid communication with the device is unoccupied.

27. The method of any of examples 22-26 wherein the operating the device in the disinfecting mode is performed according to a predetermined schedule.

28. The method of example 27 wherein the predetermined schedule corresponds to a schedule for usage of a space in fluid communication with the device, and wherein the operating the device in the disinfecting mode is performed a predetermined time increment before the usage of the space.

29. The method of any of examples 27 and 28 wherein the predetermined schedule corresponds to a recurring schedule for a space in fluid communication with the device.

30. The method of any of examples 22-29, further comprising detecting a presence in a space in fluid communication with the device, wherein the operating the device in the disinfecting mode is performed in response to detecting the presence.

31. The method of any of examples 22-30, further comprising automatically toggling the device into at least one of the disinfecting mode and a power-saving mode after completion of the chamber-cleaning mode.

32. The method of any of examples 22-31 wherein the toggling the device into the chamber-cleaning mode is performed on a recurring schedule.

33. A controller for operating a device for debilitating airborne infectious agents, the controller comprising:
one or more computer-readable storage media storing computer-executable instructions that when executed cause the controller to:
   operate the device in a disinfecting mode, wherein, in the disinfecting mode:
      an LED component of the device emits UV radiation into the airflow pathway at a first rate; and
      an air mover in the device operates at a first speed to cycle air in fluid communication with the device at a predetermined flow rate;
   toggle the device into a chamber-cleaning mode, wherein, in the chamber-cleaning mode the air mover operates at a third speed higher than the first speed.

34. The controller of example 32 wherein the instructions further cause the controller to toggle the device into a power-saving mode.

35. The controller of example 34 wherein, in the power-saving mode:
the LED component emits the UV radiation into the airflow pathway at a second rate lower than the first rate; and
the air mover operates at a third speed lower than the first speed.

36. The controller of any of examples 34 and 35 wherein, in the power-saving mode, the LED component and the air mover are not operated.

37. The controller of any of examples 34-36 wherein the instructions further cause the controller to determine that a space in fluid communication with the device is unoccupied, and wherein toggling the device into the power-saving mode is performed in response to determining the space in fluid communication with the device is unoccupied.

38. The controller of any of examples 33-37 wherein operating the device in the disinfecting mode is performed according to a predetermined schedule.

39. The controller of example 38 wherein the predetermined schedule corresponds to a schedule for usage of a space in fluid communication with the device, and wherein operating the device in the disinfecting mode is performed a predetermined time increment before the usage of the space.

40. The controller of any of examples 38 and 39 wherein the predetermined schedule is a recurring schedule.

41. The controller of any of examples 33-40 wherein the instructions further cause the controller to detect a presence in a space in fluid communication with the device, wherein operating the device into the disinfecting mode is performed in response to detecting the presence.

42. The controller of any of examples 33-41 wherein the instructions further cause the controller to automatically toggle the device into at least one of the disinfecting mode and a power-saving mode after completion of the chamber-cleaning mode.

43. The controller of any of examples 33-42 wherein the controller toggles the device into the chamber-cleaning mode on a recurring schedule.

44. The device of any of examples 3-19 wherein the multiple modes include two or more disinfecting modes.

45. The method of any of examples 22-32 wherein the disinfection mode is a first mode, wherein the predetermined flow rate is a first predetermined flow rate, and wherein the method further comprises:
operating the device in a second disinfecting mode, wherein, in the second disinfecting mode:
   the LED component emits UV radiation into the airflow pathway at a second rate to debilitate infectious agents in the airflow pathway; and
   the air mover operates at a third speed to cycle air in fluid communication with the device at a second predetermined flow rate

46. The controller of any of examples 33-43 wherein the disinfection mode is a first mode, wherein the predetermined flow rate is a first predetermined flow rate, and wherein the instructions further cause the controller to:
operate the device in a second disinfecting mode, wherein, in the second disinfecting mode:
   the LED component emits UV radiation into the airflow pathway at a second rate to debilitate infectious agents in the airflow pathway; and
   the air mover operates at a third speed to cycle air in fluid communication with the device at a second predetermined flow rate.

47. A device for debilitating airborne infectious agents as described herein.

48. A system for debilitating airborne infectious agents as described herein.

49. A method for debilitating airborne infectious agents as described herein.

We claim:

1. A device for debilitating airborne infectious agents, the device comprising:
a housing extending in an axial direction from a first end to a second end, wherein the housing includes:
an internal chamber defining an airflow pathway extending in the axial direction, the internal chamber having a surface, at least a portion of which is reflective;
a first opening toward the first end, positioned to allow air flow into the internal chamber; and
a second opening toward the second end, positioned to allow air flow out of the internal chamber; and
a light emitting diode (LED) contained in the internal chamber and positioned to emit ultraviolet (UV) radiation into the internal chamber toward the reflective surface.

2. The device of claim 1, further comprising an air mover in fluid communication with the internal chamber of the housing and positioned to direct airflow.

3. The device of claim 2 wherein:
the air mover is operable in multiple modes including an energy saving mode, a disinfecting mode, and a chamber-cleaning mode; and
the device further comprises a controller in communication with the air mover to toggle the air mover between modes.

4. The device of claim 3 wherein the controller has instructions that, when executed:
receive a signal to toggle the air mover to the disinfecting mode; and
in response to receiving the signal, send a command to the air mover, the command causing the air mover to enter the disinfecting mode, wherein the disinfecting mode includes cycling air in a predefined space at a predetermined flow rate.

5. The device of claim 3 wherein the controller has instructions that, when executed, send a command to the air mover, the command causing the air mover to enter the chamber-cleaning mode, wherein the chamber-cleaning mode includes removing dust from the interior chamber.

6. The device of claim 3, further comprising a presence detection system in communication with the controller, wherein the presence detection system is positioned to detect occupation of a predefined space in fluid communication with the device.

7. The device of claim 6 wherein the controller has instructions that, when executed:
receive a signal from the presence detection system, the signal indicating the occupation of the predefined space; and
in response to receiving the signal, send a command to the air mover, the command causing the air mover to enter the disinfecting mode, wherein the disinfecting mode is configured to cycle air in a predefined space at least four times in an hour.

8. The device of claim 1 wherein the surface of the internal chamber has a first portion and a second portion, and wherein a reflective material is disposed on the first portion of the surface and the LED is disposed on the second portion of the surface.

9. The device of claim 1 wherein the airflow pathway does not include a filter.

10. The device of claim 1 wherein:
the surface is a first surface; and
the internal chamber further comprises a second surface opposite the first surface; and
the LED is positioned to emit UV radiation in a direction from the second surface toward the first surface.

11. The device of claim 1 wherein the LED is configured to emit UV radiation having a wavelength in a wavelength range from 260 nanometers to 280 nanometers.

12. The device of claim 1, further comprising a copper screen positioned along the airflow pathway.

13. The device of claim 12, further comprising charge delivery device coupled to the copper screen to provide a biasing voltage to the copper screen.

14. The device of claim 1, further comprising one or more fins positioned within the internal chamber.

15. The device of claim 14, further comprising a charge delivery device coupled to the fins to provide a biasing voltage to the fins.

16. The device of claim 14 wherein the fins are positioned to draw heat away from an active surface of the LED.

17. The device of claim 14 wherein the LED emits UV radiation along one or more radiation travel paths, and wherein the fins are positioned to direct airflow within the airflow pathway in the internal chamber to intersect the one or more radiation travel paths.

18. The device of claim 14 wherein the fins are positioned to create an area of high pressure within the airflow pathway in the internal chamber, and wherein one or more LEDs are positioned to emit UV radiation directly into the area of high pressure.

19. A device for debilitating airborne infectious agents, the device comprising:
a housing having a first opening, a second opening spaced apart from the first opening, and an internal chamber defining an airflow pathway extending from the first opening to the second opening, wherein the internal chamber includes at least one internal surface having a reflective material;
a light emitting diode component (LED component) positioned to emit ultraviolet (UV) radiation into the internal chamber toward the reflective material on the internal surface.

20. The device of claim 19, further comprising an air mover positioned in fluid communication with the internal chamber to force air through the internal chamber along the airflow pathway

21. The device of claim 19, further comprising a controller in communication with at least one of the air mover or the LED component, the controller including a memory storing instructions that when executed cause the controller to toggle at least one of the air mover and the LED component between operational modes.

22. A method for operating a device for debilitating airborne infectious agents, the device including a housing with an internal chamber defining an airflow pathway, at least one air mover, and a light emitting diode (LED) component, the method comprising:
operating the device in a disinfecting mode, wherein, in the disinfecting mode:
the LED component emits ultraviolet (UV) radiation into the airflow pathway at a first rate to debilitate infectious agents in the airflow pathway; and
the air mover operates at a first speed to cycle air in fluid communication with the device at a predetermined flow rate;
toggling the device into a chamber-cleaning mode, wherein, in the chamber-cleaning mode the air mover operates at a second speed higher than the first speed to blow particulates out of the internal chamber.

23. The method of claim 22, further comprising toggling the device into a power-saving mode.

24. The method of claim 23 wherein, in the power-saving mode:
the LED component emits the UV radiation into the airflow pathway at a second rate lower than the first rate; and
the air mover operates at a third speed lower than the first speed.

25. The method of claim 23 wherein, in the power-saving mode, the LED component and the air mover are not operated.

26. The method of claim 22, further comprising determining that a space in fluid communication with the device is unoccupied, and wherein the toggling the device into the power-saving mode is performed in response to determining that the space in fluid communication with the device is unoccupied.

27. The method of claim 22 wherein the operating the device in the disinfecting mode is performed according to a predetermined schedule.

28. The method of claim 27 wherein the predetermined schedule corresponds to a schedule for usage of a space in fluid communication with the device, and wherein the operating the device in the disinfecting mode is performed a predetermined time increment before the usage of the space.

29. The method of claim 27 wherein the predetermined schedule corresponds to a recurring schedule for a space in fluid communication with the device.

30. The method of claim 22, further comprising detecting a presence in a space in fluid communication with the device, wherein the operating the device in the disinfecting mode is performed in response to detecting the presence.

31. The method of claim 22, further comprising automatically toggling the device into at least one of the disinfecting mode or a power-saving mode after completion of the chamber-cleaning mode.

32. The method of claim 22 wherein the toggling the device into the chamber-cleaning mode is performed on a recurring schedule.

33. A controller for operating a device for debilitating airborne infectious agents, the controller comprising:
one or more computer-readable storage media storing computer-executable instructions that when executed cause the controller to:
operate the device in a disinfecting mode, wherein, in the disinfecting mode:
an LED component of the device emits UV radiation into the airflow pathway at a first rate; and
an air mover in the device operates at a first speed to cycle air in fluid communication with the device at a predetermined flow rate;
toggle the device into a chamber-cleaning mode, wherein, in the chamber-cleaning mode the air mover operates at a third speed higher than the first speed.

34. The controller of claim 32 wherein the instructions further cause the controller to toggle the device into a power-saving mode.

35. The controller of claim 34 wherein, in the power-saving mode:
the LED component emits the UV radiation into the airflow pathway at a second rate lower than the first rate; and
the air mover operates at a third speed lower than the first speed.

36. The controller of claim 34 wherein, in the power-saving mode, the LED component and the air mover are not operated.

37. The controller of claim 34 wherein the instructions further cause the controller to determine that a space in fluid communication with the device is unoccupied, and wherein toggling the device into the power-saving mode is performed in response to determining the space in fluid communication with the device is unoccupied.

38. The controller of claim 33 wherein operating the device in the disinfecting mode is performed according to a predetermined schedule.

39. The controller of claim 38 wherein the predetermined schedule corresponds to a schedule for usage of a space in fluid communication with the device, and wherein operating the device in the disinfecting mode is performed a predetermined time increment before the usage of the space.

40. The controller of claim 38 wherein the predetermined schedule is a recurring schedule.

41. The controller of claim 33 wherein the instructions further cause the controller to detect a presence in a space in fluid communication with the device, wherein operating the device into the disinfecting mode is performed in response to detecting the presence.

42. The controller of claim 33 wherein the instructions further cause the controller to automatically toggle the device into at least one of the disinfecting mode and a power-saving mode after completion of the chamber-cleaning mode.

43. The controller of claim 33 wherein the controller toggles the device into the chamber-cleaning mode on a recurring schedule.
